(19) United States
(12) Patent Application Publication
(10) Pub. No.: US 2006/0100168 A1
(43) Pub. Date: May 11, 2006

(54) Method of treating cancer using adenosine and its analogs

(75) Inventors: Katya Ravid, Chestnut Hill, MA (US); Jun Lu, Medford, MA (US)

Correspondence Address: Ronald I Eisenstein;Nixon Peabody, 100 Summer Street, Boston, MA, 02110, US

(73) Assignee: The Trustee of Boston University, Boston, MA (US)

(21) Appl. No.: 10/529,524

(22) PCT Filed: Sep. 30, 2003
(86) PCT No.: PCT/US03/30701

§ 371 Date: Aug. 19, 2005

Related U.S. Application Data
(60) Provisional application No. 60/414,706, filed on Sep. 30, 2002.

(51) Int. Cl. A61K 31/7076; A61K 31/56; A61K 31/137

(52) U.S. Cl. 514/46; 514/182; 514/651

(57) ABSTRACT

The present invention provides methods of treating individuals having malignancies associated with estrogen receptor activity comprising administering to an individual affected with said malignancy, an effective amount of adenosine analog in a pharmaceutical carrier to downregulate or diminish estrogen receptors in the cells. The invention further provides methods of identifying novel adenosine analogues capable of treating malignant cells expressing estrogen receptors. The invention also provides kits comprising adenosine analogs for downregulating estrogen receptors in cells and kits for screening for novel adenosine analogs capable of downregulating estrogen receptors. Further, the invention provides uses of adenosine analogs in downregulation of estrogen receptors, cell growth and cell cycle, as well as pharmaceutical compositions comprising adenosine analogs effective in suppressing cellular growth, cell cycle or downregulating estrogen receptors.

This application claims benefit under 35 U.S.C. §119(e) of 60/414,706 filed Sep. 30, 2002.

This invention was supported by National Institutes of Health grant No. CA79397 and the government of the United States has certain rights thereto.

### FIELD OF THE INVENTION

The present invention is directed to a method of treating estrogen-receptor positive cancers comprising administering to an individual in need thereof adenosine receptor agonists that are capable of downregulating estrogen receptors. Preferably the cancer is breast cancer.

### BACKGROUND OF THE INVENTION

The human estrogen receptor (ER) is a member of the nuclear receptor superfamily of transcription factors (Evans, Science 240:889-895 (1988)). Upon binding a ligand, ER undergoes a conformational change initiating a cascade of events ultimately leading to its association with specific regulatory regions within target genes (O'Malley et al., Hormone Research 47:1-26 (1991)). The ensuing effect on transcription is influenced by the cell and promoter context of the DNA-bound receptor (Tora et al. Cell 59:471-487 (1989), Tasset et al., Cell 62:1177-1181 (1990); McDonnell et al. Mol. Endocrinol. 9:659-669 (1995); Tzukerman et al. Mol. Endocrinol. 8:21-30 (1994)). It is in this manner that the physiological ER-agonist, estradiol, exerts its biological activity in the reproductive, skeletal and cardiovascular systems (Clark and Peck, Female Sex Steroids:Receptors and Function (eds) Monographs Springer-Verlag, New York (1979); Chow et al., J. Clin. Invest. 89:74-78 (1992); Eaker et al. Circulation 88:1999-2009 (1993)).

Approximately 180,000 women are diagnosed with breast cancer each year in the United States. Most of these women are treated using surgery and local radiotherapy. However, nearly 60,000 women still go on to develop metastatic breast cancer each year, and about 45,000 of these patients eventually die from their malignancies. While metastatic breast cancer is rarely curable, it is treatable with modern pharmaceuticals that can prolong patient survival and reduce the morbidity associated with metastatic lesions. Foremost among these therapies are hormonal manipulations that include selective estrogen receptor modifiers (SERMs). SERMs are small ligands of the estrogen receptor that are capable of inducing a wide variety of conformational changes in the receptor and thereby eliciting a variety of distinct biological profiles. SERMs not only affect the growth of breast cancer tissue but also influence other physiological processes. The most widely used SERM in breast cancer is tamoxifen, which is a partial estrogen receptor agonist/antagonist that produces objective responses in approximately 50% of the patients. Unfortunately, almost all patients who take tamoxifen eventually relapse with tamoxifen-resistant tumors. Approximately half of the patients who fail tamoxifen treatment will respond to a subsequent hormonal manipulation therapy such as ovariectomy, aromatase inhibitors, or other SERMs. The second line therapies for hormonal manipulation therapy of metastatic breast cancer represent a substantial unmet need because no single agent has become the treatment of choice for patients who fail tamoxifen therapy. The ideal agent would be a medication that induces regression of metastatic breast cancer lesions in women who have previously responded to tamixofen therapy.

SERMs modulate the proliferation of uterine tissue, skeletal bone density, and cardiovascular health, including plasma cholesterol levels. In general, estrogen stimulates breast and endometrial tissue proliferation, enhances bone density, and lowers plasma cholesterol. Many SERMs are bifunctional in that they antagonize some of these functions while stimulating others. For example, tamoxifen, which is a partial agonist/antagonist of estrogen receptor, inhibits estrogen-induced breast cancer cell proliferation but stimulates endometrial tissue growth and prevents bone loss.

Estrogen has also been shown to function as a mitogen in estrogen-receptor (ER) positive breast cancer cells. Thus, treatment regiments which include antiestrogens, synthetic compounds which oppose, the actions of estrogen have been effective clinically in halting or delaying the progression of the disease (Jordan and Murphy, Endocrine Reviews 11:578-610 1990); Parker, Breast Cancer Res. Treat. 26:131-137 (1993)).

One of the most studied estrogen receptor function interfering compounds is tamoxifen (TAM), (Z)1,2-diphenyl-1-[4-[2-(dimethylamino) ethoxy]phenyl]-1-butene, (Jordan and Murphy, Endocrine Reviews 11:578-610 (1990)). As discussed above, tamoxifen functions as an antagonist in most ER-positive tumors of the breast and ovum, but displays a paradoxical agonist activity in bone and the cardiovascular system and partial agonist activity in the uterus (Kedar et al. Lancet 343:1318-1321 (1994); Love et al., New Engl. J. Med. 326:852-856 (1992); Love et al., Ann. Intern. Med. 115:860-864 (1991)). Thus, the agonist/antagonist activity of the ER-tamoxifen complex is influenced by cell context. This important observation is in apparent contradiction to longstanding models that hold that ER only exists in the cell in an active or an inactive state (Clark and Peck, Female Sex Steroids:Receptors and Functions (eds) Monographs on Endocrinology, Springer-Verlag, New York (1979)). Rather it indicates that different ligands acting through the same receptor can have different biological effects in different cells. Definition of the mechanism of this selectivity is likely to advance the understanding of processes such as tamoxifen resistance, observed in most ER-containing breast cancers, where abnormalities in ER-signaling are implicated (Tonetti and Jordan, Anti-Cancer Drugs 6:498-507 (1995)).

Tamoxifen, as well as a structurally similar compound known as 4-OH-tamoxifen, raloxifene, and ICI 164,384 have been developed for the treatment and/or prevention of osteoporosis, cardiovascular disease and breast cancer in addition to the treatment and/or prevention of a variety of other disease states. Both compounds have been shown to exhibit an osteoprotective effect on bone mineral density combined with a positive effect on plasma cholesterol levels and a greatly reduced incidence of breast and uterine cancer. Unfortunately, tamoxifen and raloxifene both have unacceptable levels of life-threatening side effects such as endometrial cancer and hepatocellular carcinoma. Therefore, there is a need for new breast cancer therapies.

### SUMMARY OF THE INVENTION

It is therefore the purpose of the present invention to provide a novel method for treating individuals affected with cancers associated with estrogen receptor expression, such as estrogen receptor positive cancers, including breast and ovarian cancers.

In one embodiment, the invention provides a method of treating breast cancer in an individual in need thereof by administering an effective amount of at least one adenosine analog and a pharmaceutically acceptable carrier to decrease estrogen receptors.

Estrogen receptors according to the present invention include estrogen receptor alpha and estrogen receptor beta. In one preferred embodiment, the estrogen receptor is estrogen receptor alpha.

The purine nucleoside adenosine is a natural metabolite that plays a role in several physiologic and pathologic processes, such as inhibition of platelet aggregation, cardioprotection after ischemia, vasodilation, mast cell activation and lypolysis (see review (1)). Adenosine is produced and released at micromolar concentration in/from several tissues, such as fibroblasts, endothelial cells, epithelial cells, cardiac myocytes, muscle cells, and platelets (2-5). The level of adenosine is further elevated under conditions such as muscle exercise (6), or ischemia (7).

Adenosine exerts many of its effects by activation of specific cell surface receptors. To date, four adenosine receptors (AR), the A1AR, A2aAR, A2bAR and A3AR have been cloned (8, 9). Medicinal chemistry has provided different adenosine analogs that are potent selective activators of specific adenosine receptors. These include agonists, such as 2-Chloro-N⁶-cyclopentyladenosine (CCPA) (A1AR selective), 2-p-(2-Carboxyethyl)phenethylamino-5′-N-ethylcarboxamidoadenosine CGS-21680 (A2aAR selective), N⁶-(3-iodobenzyl)adenosine-5′-N-methyluronamide (IB-MECA) (A3AR selective) and 5′-(N-Ethylcarboxamido)adenosine (NECA) (activates both A2aAR and A2bAR).

Adenosine and its analogues were recently shown to inhibit growth or induce apoptosis in several types of cancer cells. Epidermoid carcinoma A431 cells and some human cancer cells were inhibited by agonists for A1AR or A2AR (10-12). HL-60 leukemia and U-937 lymphoma cells were reported to be induced into apoptosis by A3AR agonists (13, 14). Fishman et al found that adenosine is one active component within skeletal muscle cell-conditioned medium, which can inhibit the growth of SK-28 melanoma cells, K-562 chronic myelogenous leukemia cells, and MCF-7 breast cancer cells (15).

Preferably, the estrogen receptor down-regulating adenosine analog or derivative thereof is selective to the A3 adenosine receptor (A3AR). In one preferred embodiment, the adenosine analog is selected from a group consisting of N6-(3-iodobenzyl) adenosine-5′-N-methyluronamide (IB-MECA), 2-chloro-deoxyadenosine (CdA), 3′-deoxyadenosine (Cordycepin), 2-chloro-N-6-cyclopentyladenosine (CCPA), 5′-(N-Ethylcarboxamido) adenosine (NECA), 2-chloro-adenosine (CADO), inosine (INO) or a derivative or a combination thereof.

In one preferred embodiment, the adenosine analog useful according to the present invention is IB-MECA, CdA, Cordycepin or a derivative or a combination thereof.

In the most preferred embodiment, the estrogen receptor down-regulating adenosine analog is IB-MECA or a functional, estrogen receptor down-regulating derivative thereof. Preferably, the estrogen receptor is estrogen receptor alpha.

Estrogen receptors are known to be expressed in various human tissues including reproductive tissues such as ovaries, uterine, vagina, and testicles (for review, see, e.g. OMIM at http://www.ncbi.nlm.nih.gov/entrez). These receptors are also present in some pituitary adenomas and osteosarcomas. The estrogen receptor expression in mammary glands and their relationship with breast cancer has been widely studied.

Two isoforms of human estrogen receptor, ER-alpha (ESRA, OMIM ID. No. 133430; GenBank ID Nos. gi:182192 and gi:31233) and ER-beta (ESRB, OMIM ID No. 601663, GenBank ID Nos. gi:2911151 and gi:34193698), have a distinct, although sometimes overlapping expression pattern. Further, additional ESR isoforms, generated by alternative mRNA splicing, have been defined in several tissues and they are postulated to play a role in tumorigenesis or in modulating the estrogen response (OMIM entry No. 601663, at http://www.ncbi.nlm.nih.gov/entrez). The present invention contemplates downregulating estrogen receptors in general. In one preferred embodiment, the estrogen receptor is estrogen receptor alpha.

An individual in need of treatment may have any malignancy which is associated with estrogen receptor mediated growth. Such malignancies include, but are not limited to breast tumors, osteosarcomas (Chaidarun, et al., Molec. Endocr. 12: 1355-1366, 1998), pituitary adenomas (Shupnik, et al., J. Clin. Endocr. Metab. 83: 3965-3972, 1998) as well as cancers of human reproductive organs expressing estrogen receptors including ovaries, uterus, and testicles, particularly in the Leydig cells.

In one preferred embodiment of the present invention, the adenosine analog down-regulates estrogen receptor levels in the transcript level. Therefore, the invention is particularly useful in treating malignancies which are caused by mutated and/or truncated estrogen receptors that activate transcription even in the absence of estrogen, and cannot therefore be inhibited with pharmaceutical compounds functioning as estrogen analogs.

The estrogen receptor down-regulating analogue according to the present invention also includes mixtures of different estrogen receptor down-regulating analogues.

In a preferred embodiment, the individual in need of treatment by adenosine analogs is affected with an estrogen receptor alpha (ERalpha) positive cancer, such as breast cancer including ductal carcinoma in situ (DCIS), infiltrating (or invasive) ductal carcinoma (IDC), or infiltrating (or invasive) lobular carcinoma (ILC).

Examples of ERalpha positive cells useful according to the present invention include, but are not limited to breast cancer cell (BCC) lines including but not limited to MCF-7 (high amount), T-47D, ZR-75, CAMA-1, BT483, BT474, MDA-MB-361, and MDA-MB-134.

Non-exclusive examples of estrogen receptor beta positive cells include breast tumor cells, ovarian tumor cells (Chu, S. et al., Estrogen receptor isoform gene expression in ovarian stromal and epithelial tumors. J. Clin. Endocr. Metab. 85: 1200-1205, 2000), and pituitary adenomas including prolactinomas, mixed growth hormone/prolactine tumors, gonadotroph tumors, and somatotroph, corticotroph, and null cell tumors (Chaidarun, S. S. et al., Differential expression of estrogen receptor-beta (ER-beta) in human pituitary tumors: functional interactions with ER-alpha and a tumor-specific splice variant. J. Clin. Endocr. Metab. 83: 3308-3315, 1998).

Further, any malignant cell type which can be shown to express estrogen receptors using either protein or mRNA expression, using method well known to one skilled in the art, is considered to be a target malignancy for the methods of the present invention.

In one embodiment, the method of the present invention comprises administering ERalpha down-regulating agonists before, after or simultaneously with tamoxifen ((Z)1,2-diphenyl-1-[4-[2-(dimethylamino) ethoxy]phenyl]-1-butene), 4-OH-tamoxifen (4-OH-(Z)1,2-diphenyl-1-[4-[2-(dimethylamino) ethoxy]phenyl]-1-butene), raloxifene, and ICI 164,384 (N-(n-butyl)-11-[3,17β-dihydroxyestra-1,3,5(10)-trien-7α-yl]N-methylundecanamide).

In one embodiment, the invention provides a method of treating breast cancer with an estrogen receptor alpha mutation Tyr 537 to Asn (T 1609 A), by administering an estrogen receptor down-regulating amount of an adenosine analog to the individual with cells having the mutation. This mutation has been identified in approximately 1 of 30 metastatic breast cancers (http://www.ncbi.nlm.nih.gov/entrez/dispomim.cgi?id=133430). This substitution confers constitutive transcriptional activity to estrogen receptor and its activity cannot be antagonized with antiestrogens such as tamoxifen and pure antiestrogen ICI 164384 (Zhang Q. X. et al., Cancer Res., 1997, April 1; 57(7):1244-9).

In one embodiment, the invention provides a method of identifying novel compounds useful for down-regulating estrogen receptors. In this way, one can identify compounds, including adenosine analogs and derivatives thereof, useful for treating estrogen-receptor positive cancers. The method comprises the steps of contacting an ERalpha or estrogen receptor beta (ERbeta) positive cell with a test compound and calculating cell growth, measuring ERalpha or ERbeta levels by western blot analysis and/or quantitative RT-PCR, and determining cell cycle arrest by flow cytometry analysis. Cells and cell lines useful according to this embodiment include cell lines expressing ERalpha, such as MCF-7, T-47D, ZR-75, CAMA-1, BT483, BT474, MDA-MB-361, and MDA-MB-134.

In one preferred embodiment, the method comprises administering a test compound to cells and detecting the level of ER transcripts from the cells. If the ER transcript level is decreased compared to the same cells grown in the absence of the test compound, the test compound is considered to have an ER down-regulating activity. In one embodiment the ER is ERalpha. In an alternative method the ER is ERbeta.

The invention further provides kits for downregulating estrogen receptors, kits for detecting novel estrogen receptor downregulating adenosine analogs, and uses to of adenosine analogs to downregulate estrogen receptors, cell growth and cell cycle, and pharmaceutical compositions comprising adenosine analogs to downregulate cell growth, cell cycle and/or estrogen receptor level in the cells.

### BRIEF DESCRIPTION OF THE DRAWINGS

FIG. 1 shows the chemical structures of adenosine and adenosine analogs.

FIGS. 2A-2E show the effects of adenosine and adenosine receptor agonists on MCF-7 cell colony formation. MCF-7 cells were plated in soft agar and treated with adenosine (FIG. 1A), CCPA (FIG. 1B), CGS21680 (FIG. 1C), NECA (FIG. 1D), or IB-MECA (FIG. 1E), with the indicated concentrations. After two weeks of treatment, colony numbers were counted and expressed as the percentage of those of vehicle-treated cells (0 μM). Data shown are averages of triplicate experiments and error bars represent standard deviations.

FIGS. 3A-3C show the effect of IB-MECA on colony formation, growth and apoptosis of different breast cancer cell lines. In FIG. 3A, human cancer cell lines MCF-7, ZR-75, T47D, Hs578T and HeLa were plated in soft agar and treated with 100 μM IB-MECA. Numbers of colonies formed were determined after two weeks in culture, and expressed as the percentage of those of vehicle-treated cells (DMSO). In FIG. 3B, MCF-7, ZR-75, T47D and Hs578T cells were plated in 6 well plates, and treated with 100 μM of IB-MECA for three days. Cell numbers were counted and expressed as percentages of cell counts before treatment (Day 0). In FIG. 3C, MCF-7, ZR-75, T47D and Hs578T cells were treated with 100 μM IB-MECA for two days. Cells were stained with propidium iodide and subjected to FACS analyses. Apoptotic events were determined by quantification of the sub-2n populations on fluorescence histograms, and were expressed as the percentage of total events. All data shown are averages of triplicate experiments, and error bars represent standard deviations.

FIGS. 4A-4D show that IB-MECA induces growth inhibition and downregulates cyclins in MCF-7 cells. In FIG. 4A, MCF-7 cells were treated with vehicle (DMSO) or 100 μM IB-MECA and were counted after 1, 2 or 3 days. The number of cells was expressed as the percentage of cell count before treatment (Day 0). Data shown are averages of triplicate experiments and error bars represent standard deviations. In FIG. 4B, MCF-7 cells were treated with vehicle (DMSO) or 100 μM IB-MECA for 2 days. Cells were stained with propidium iodide (PI) and subjected to FACS analyses. The percentages of cells in different phases of the cell cycle were as follows: G1 phase: 50.2% (DMSO) and 64.2% (IB-MECA); S phase: 25.2% (DMSO) and 12.3% (IB-MECA); G2/M: 24.6% (DMSO) and 23.4% (IB-MECA). These calculations represent averages of 3 determinations. Representative fluorescence histograms are shown. In FIG. 4C, MCF-7 cells were treated with 100 μM IB-MECA for the indicated times. Cells were harvested and subjected to Western blot analyses using indicated antibodies. In FIG. 4D, MCF-7 cells were treated with different dosages of IB-MECA or NECA and harvested after 48 hours. Cells were subjected to Western blot analyses using indicated antibodies.

FIGS. 5A and 5B show that the effect of IB-MECA is not through activation of the A3 adenosine receptor. MCF-7 cells were stably transfected with human A3 adenosine receptor cDNA. In FIG. 5A, the expression of A3 adenosine receptor in MCF-7 cells or a pool of stably transfected cells (MCF-7+A3) was assayed by RT-PCR. Reverse transcription reactions were performed with (+) or without (−) reverse transcriptase, followed by PCR reactions using primers specific for A3 adenosine receptor (A3AR) or GAPDH. Representative agarose gel pictures are shown. In FIG. 5B, MCF-7 cells or pool of MCF-7 cells stably expressing A3 adenosine receptor (MCF-7+A3) were plated into soft agar and treated with different concentrations of IB-MECA. Colony numbers were determined after two weeks in culture and expressed as those of vehicle-treated cells. Data shown are averages of triplicate experiments and error bars represent standard deviations.

FIGS. 6A-6D show how that IB-MECA treatment downregulates estrogen receptor α mRNA level, protein level and transcriptional activity in MCF-7 cells. In FIG. 6A, MCF-7 cells were treated with vehicle (−) or 100 μM IB-MECA (+) for the indicated times. Reverse transcription reactions were carried out with (+RT) or without (−RT) reverse transcriptase, using RNA isolated from the samples. Primers specific for estrogen receptor α and GAPDH were used in semi-quantitative PCR reactions. Pictures of RT-PCR products analyzed on agarose gels are shown. In FIG. 6B, estrogen receptor α (ERα), cyclins and actin (loading control) were assayed with Western blot analyses, using indicated antibodies, after MCF-7 cells were treated with 100 μM IB-MECA for the indicated times. In FIG. 6C, MCF-7 cells were treated with different concentrations of IB-MECA or NECA for two days. Cells were harvested and subjected to Western blot analyses with antibodies against ERα or actin. In FIG. 6D, MCF-7 cells transfected with pERE-Tk-Luc or pCMV-β-Gal plasmids were treated with vehicle (O) or indicated concentrations of IB-MECA for 12 hours. Cells were harvested and reporter gene activity was assayed as detailed in Methods. Data shown are averages of triplicate experiments and error bars represent standard deviations.

FIGS. 7A and 7B show that overexpression of estrogen receptor a rescues growth inhibition by IB-MECA in MCF-7 cells. MCF-7 cells were transiently transfected with pcDNA3-ERα (pERα) or pcDNA3 (vector) with a transfection efficiency of approximately 40% (see Methods). Cells were treated with vehicle (DMSO) or 100 μM IB-MECA for one day. In FIG. 7A, expression of estrogen receptor α (ERα) was determined by Western blot analysis. Actin served as a loading control. In FIG. 7B, cell numbers were determined post one day incubation, and expressed as the percentage of cell count before treatment (Day 0). Data represent averages of triplicate experiments and error bars represent standard deviations. Samples labeled with “*” showed a p value of less than 0.002 under Student's t-test.

FIGS. 8A-8E show the effects of IB-MECA on mRNA level and mRNA half-life of estrogen receptor α in MCF-7 cells. FIG. 8A shows the mRNA levels of estrogen receptor a (ERα), pS2 and estrogen receptor β (ERβ) in IB-MECA treated cells. MCF-7 cells were treated with vehicle (−) or 100 μM IB-MECA (+) for the indicated periods. Reverse transcription reactions were carried out on total RNA isolated from the samples (same samples as in FIG. 6B). Primers specific for ERα, pS2, ERβ and GAPDH were used in semi-quantitative PCR reactions. Representative pictures of RT-PCR products analyzed on poly-acrylamide gels are shown. FIG. 8B shows results of the experiment wherein after 30-minute pre-incubation with 50 μg/ml of the protein synthesis inhibitor cycloheximide, MCF-7 cells were treated with vehicle (DMSO) or 100 μM IB-MECA for the indicated hours (hr). Cells were harvested and assayed for ERα or actin contents with Western blot analyses. FIG. 8C shows the ERα mRNA half-life in IB-MECA treated cells. MCF-7 cells were pre-treated with vehicle (DMSO, −) or 100 μM IB-MECA (+) for 6 hours before adding the transcription inhibitor DRB (80 μM). Cells were harvested after indicated periods, and were subjected to RT-PCR analyses using specific primers for ERα. Total RNA samples of 2 μg each were resolved on a denaturing agarose gel and the 18S rRNA bands were used as loading controls. FIG. 8D demonstrates results from a representative experiment illustrateing the linear range of PCR reactions. Indicated template amounts of the 0 hour DMSO-treated sample in (FIG. 8C) were amplified. A representative picture of PCR products analyzed on an acrylamide gel is shown. Intensities of the bands were quantitated using Kodak Digital Scientific 1D software and presented in arbitrary units (AU). Data shown are averages of two PCR reactions and error bars represent variations. A linear regression fitting curve was plotted with R² value of 1. FIG. 8E shows that mRNA half-lives as quantitated for samples in (FIG. 8C). Average intensities of duplicate PCR reaction products (for ERα) were normalized with corresponding intensities of 18S rRNA. Normalized ERα data were presented as the percentage of the level at 0 hour time point, and plotted on a logarithmic scale. Data shown are averages of three independent experiments and error bars represent standard deviations.

### DETAILED DESCRIPTION OF THE INVENTION

The present invention provides a method of treating individuals having malignancies associated with estrogen receptor activity comprising administering to an individual affected with said malignancy, an effective amount of adenosine analog in a pharmaceutical carrier to downregulate or diminish estrogen receptors in the cells. Preferably, the malignancy is breast cancer or ovarian cancer.

The invention is based upon a surprising finding that adenosine analogs diminish, or downregulate the amount of estrogen receptors and estrogen dependent cancer cell growth.

In a preferred embodiment, the adenosine analog is selected from the group consisting of N6-(3-iodobenzyl) adenosine-5′-N-methyluronamide (IB-MECA), 2-chloro-deoxy-adenosine (CdA), 3′-deoxyadenosine (Cordycepin), 2-chloro-N-6-cyclopentyladenosine (CCPA), 5′-(N-Ethylcarboxamido) adenosine (NECA), 2-chloro-adenosine (CADO), inosine (INO), which all can be purchased from Sigma (St. Louis, Mo.). More preferably, the adenosine analog is an A3 adenosine receptor selective analog, for example, IB-MECA.

The term “treatment” as used throughout the specification means: (1) preventing such disease from occurring in a subject who may be predisposed to these diseases but who has not yet been diagnosed as having them; (2) inhibiting these diseases, i.e., arresting or slowing down their development; or (3) ameliorating or relieving the symptoms of these diseases.

The term “effective amount” as used throughout the specification means an amount of the compound necessary to obtain a detectable therapeutic effect. The therapeutic effect may include, for example, without limitation, inhibiting the growth of undesired tissue or malignant cells, inhibition of tumor cell growth, decreased levels of an estrogen receptor transcript or protein. Estrogen receptors include estrogen receptor alpha (or ESR1, OMIM ID No. 13340, at http://www.ncbi.nlm.nih.gov) and estrogen receptor beta (or ESR2, OMIM ID No. 601663, at http://www.ncbi.nlm.nih.gov), and the like. The precise effective amount for a subject will depend upon the subject's size and health, the nature and severity of the condition to be treated, and the like. Thus, it is not possible to specify an exact effective amount in advance. However, the effective amount for a given situation can be determined by routine experimentation based on the information provided herein.

Individuals who can be treated with the methods of the present invention include those affected with an estrogen receptor associated cancers including osteosarcomas, pituitary adenomas, testicular, uterine, ovarian and breast cancers. Different types of breast cancers include, but are not limited to ductal carcinoma in situ (DCIS), infiltrating (or invasive) ductal carcinoma (IDC), or infiltrating (or invasive) lobular carcinoma (ILC). In one preferred embodiment, the individual is affected with breast cancer wherein the cancer cells are estrogen receptor (ER) positive. In one preferred embodiment, the ER is ERalpha. One preferred group of individuals treated by the present invention are those having tumors containing cells that exhibit anchorage independent growth.

In another preferred embodiment, the individual affected with breast cancer which is unresponsive to tamoxifen, 4-OH-tamoxifen, raloxifene, or ICI 164,384 therapy.

For therapeutic applications, the compounds may be suitably administered to the individual affected with cancer, alone or as part of a pharmaceutical composition, comprising the compounds together with one or more acceptable carriers thereof and optionally other therapeutic ingredients. The carrier(s) must be “pharmaceutically acceptable” in the sense of being compatible with the other ingredients of the formulation and not deleterious to the recipient thereof. In one embodiment, the adenosine analog of the present invention is administered together with tamoxifen, 4-OH-tamoxifen, raloxifene, or ICI 164,384, or a mixture thereof.

The pharmaceutical compositions of the present invention include those suitable for oral, rectal, nasal, (including buccal and sublingual), vaginal, parenteral (including subcutaneous, intramuscular, intravenous and intradermal), occular using eye drops, transpulmonary using aerosolubilized or nebulized drug administration. The formulations may conveniently be presented in unit dosage form, e.g., tablets and sustained release capsules, and in liposomes, and may be prepared by any methods well know in the art of pharmacy. (See, for example, Remington: The Science and Practice of Pharmacy by Alfonso R. Gennaro (Ed.) 20th edition, Dec. 15, 2000, Lippincott, Williams & Wilkins; ISBN: 0683306472.)

When preparing the pharmaceutical composition of the present invention, such preparative methods include the step of bringing into association with the adenosine analog or a derivative thereof ingredients such as the carrier which constitutes one or more accessory ingredients. In general, the compositions are prepared by uniformly and intimately bringing into association the active ingredients, including the adenosine analogs, with liquid carriers, liposomes or finely divided solid carriers or both, and then if necessary shaping the product.

Compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

It will be appreciated that actual preferred amounts of a given compound used in a given therapy will vary according to the particular adenosine analog compound being utilized, the particular compositions formulated, the mode of application, the particular site of administration, the patient's weight, general health, sex, etc., the particular indication being treated, etc. and other such factors that are recognized by those skilled in the art including the attendant physician. Optimal administration rates for a given protocol of administration can be readily determined by those skilled in the art using conventional dosage determination tests.

In one embodiment, the invention provides a pharmaceutical composition for suppressing cell cycle and/or cellular growth comprising an effective amount of at least one adenosine analog and a pharmaceutically acceptable carrier. In one preferred embodiment, the adenosine analog is selected from the group consisting of A3 receptor binding analog, IB-MECA, 2-chloro-adenosine, and estrogen receptor downregulating derivatives thereof.

A method of identifying ER inhibitory compounds, including adenosine analogs, useful for the treatment of cancer, such as breast or ovarian cancer, comprises the steps of treating cancer cells with the adenosine analog in question and calculating cell growth, measuring ERalpha levels by western blot analysis and/or quantitative RT-PCR, and determining cell cycle arrest by flow cytometry analysis.

In another preferred embodiment, this treatment is combined with another form of cancer therapy including use of SERMS such as tamoxifen, radiation, a chemotherapeutic, an antiangiogenic agent, etc. Anti-angiogeneic agents are known to one skilled in the art and include, but are not limited to VEGF and its receptors (Kim et al., Nature 362:841-844, 1993; Saleh et al., Cancer Res 56:393-401, 1996; Millauer et al., Cancer Res 56:1615-1620, 1996; Millauer et al., Nature 367:576-579, 1994; Strawn et al., Cancer Res 56:3540-3545, 1996; VEGF antagonists (Claffey et al., Cancer Res 56:172-181, 1996); both human and murine forms of angiostatin, a proteolytic fragment of plasminogen (O'Reilly et al., Cell 79:315-28, 1994; O'Reilly et al., Nat Med 2:689-92, 1996). Similarly, a C-terminal fragment of collagen XVIII, termed endostatin, has been reported to exhibit anti-angiogenic and tumor-regressing activities accompanied by a lack of acquired tumor resistance (O'Reilly et al., Cell 88:277-85, 1997; Boehm et al., Nature 390:404-7, 1997); and vector-mediated delivery of angiostatin, endostatin, soluble Flt1 ectodomains, and soluble neuropilin (sNRP) domains, (see, e.g., Takayama et al., Cancer Res 60:2169-77, 2000; Griscelli et al., Proc Natl Acad Sci USA 95:6367-6372, 1998; Blezinger et al., Nat Biotechnol 17:343-8 1999; Chen et al., Cancer Res 59:3308-3312, 1999; Sauter et al., Proc Natl Acad Sci USA 97:4802-4807, 2000; Feldman et al., Cancer Res 60:1503-1506, 2000).

The invention further provides a use of pharmaceutical compounds comprising adenosine analogs, such as A3 adenosine receptor agonists, IB-MECA, 2-chloro-adenosine and derivatives thereof, for treatment of cancer. The cancer preferably comprises cells expressing estrogen receptors, most preferably ERalpha. The most preferred treatment targets are breast cancer and ovarian cancer. In one embodiment, the cancer comprises cells growing anchorage independently.

The present invention also provides kits for detecting or screening cancer treatment compounds capable of downregulating estrogen receptors. Such kits typically comprise two or more components necessary for performing a screening assay of compounds that are capable of downregulating estrogen receptors and therefore useful in treatment of cancers. Components may be compounds, cells, reagents, containers and/or equipment. For example, one container within a kit may contain a monoclonal antibody or fragment thereof that specifically binds estrogen receptor to enable detection of downregulation of estrogen receptors in the cells. Such antibodies or fragments may be provided attached to a support materials known to one skilled in the art. One or more additional containers may enclose elements, such as reagents or buffers, to be used in the assay. Such kits may also, or alternatively, contain a detection reagent as described above that contains a reporter group suitable for direct or indirect detection of antibody binding.

In one preferred embodiment, the kit is designed to detect and measure estrogen receptor mRNA level. Such kits generally comprise at least one oligonucleotide probe or primer, that hybridizes to a polynucleotide encoding estrogen receptor protein(s). Such an oligonucleotide may be used, for example, within a reverse transcriptase (RT)-PCR, PCR or hybridization assay. Additional components that may be present within such kits include a second oligonucleotide and/or a diagnostic reagent or container to facilitate the detection of a polynucleotide encoding an estrogen receptor protein. Primers may also be labeled to enhance detection.

The kits provided by the present invention also include at least one control reagent, such as IB-MECA, or other adenosine analog downregulating estrogen receptors. Such control reagent is provided so that it can be administered to the cells expressing estrogen receptors provided in the kit, and thereby allow comparison of test compound(s) to an effective estrogen receptor downregulating agent, and consequently provide a reference point for effectiveness of the novel test compound in downregulating estrogen receptors. The kit also provides instructions how to measure estrogen receptor downregulation, for example, as provided by the examples shown in this specification.

Means for detecting estrogen receptor downregulation include, for example immunological techniques using estrogen receptor antibodies. Preferably, the detection means include techniques based on detection of mRNA levels such as RT-PCR based methods include, but are not limited to PYROSEQUENCING™ (Uppsala, Sweden); real-time PCR systems which rely upon the detection and quantitation of a fluorescent reporter, the signal of which increases in direct proportion to the amount of PCR product in a reaction, for example TaqMan® (ABI 7700 (TaqMan®), Applied BioSystems, Foster City, Calif.); hybridization-based techniques; an INVADER® assay (Third Wave Technologies, Inc (Madison, Wis.)), fluorescence-based PCR quantification techniques, solid-phase minisequencing (U.S. Pat. No. 6,013,431 and in Wartiovaara and Syvanen, Quantitative analysis of human DNA sequences by PCR and solid-phase minisequencing. Mol Biotechnol 2000 June; 15(2):123-131); and MALDI-TOF mass array (Sequenom's MassArray™ system).

Test compounds may include small organic or inorganic molecules, libraries of molecules, phage display libraries and the like known to one skilled in the art. For, example, synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries can be produced, if desired, according to methods known in the art, e.g., by standard extraction and fractionation methods. Furthermore, if desired, any library or compound is readily modified using standard chemical, physical, or biochemical methods.

It is to be understood that while the invention has been described in conjunction with the preferred specific embodiments thereof that the foregoing description as well as the examples that follow are intended to illustrate and not limit the scope of the invention. Other aspects, advantages and modification within the scope of the invention will be apparent to those skilled in the art to which the invention pertains.

### EXAMPLE

We have found that IB-MECA, an A₃AR agonist, can potently inhibit cell proliferation in both anchorage-independent and anchorage-dependent assays. Our results indicated that the effect of IB-MECA in ERα-positive breast cancer cells was not mediated by the activation of A₃AR, but rather involved ERα downregulation. These results point to the potential use of IB-MECA and its derivaties in the treatment of estrogen receptor positive cancers, and demonstrate the existence of a signaling pathway initiated by IB-MECA and its derivatives, that can regulate ERα and ERα-mediated processes.

Methods

Chemicals: All chemicals were purchased from Sigma (St Louis, Mo.), unless otherwise indicated. N⁶-(3-iodobenzyl)adenosine-5′-N-methyluronamide (IB-MECA) was purchased from Sigma or from Tocris (Avonmouth, UK), in order to examine two different batches of preparation. IB-MECA, 2-Chloro-N⁶-(3-iodobenzyl)adenosine-5′-N-methyluronamide (C1-IB-MECA), 5′-(N-Ethylcarboxamido)adenosine (NECA) and 2-Chloro-N⁶-cyclopentyladenosine (CCPA) were dissolved in DMSO, with a stock concentration of 50 mM, and aliquoted and stored in −80° C. Adenosine was freshly dissolved before experiments into whole cell culture medium. 2-p-(2-Carboxyethyl)phenethylamino-5′-N-ethylcarboxamidoadenosine (CGS21680) was dissolved in phosphate buffered saline (PBS) (Invitrogen, Carlsbad, Calif.) at 2 mM.

Plasmids: pERE-Tk-Luc, consisting of a promoter containing estrogen responsive elements, driving the luciferase reporter gene (39), and pcDNA3-ERα, consisting of the CMV promoter driving the expression of human estrogen receptor cDNA (39) were kind gifts from Dr. Zhixiong Xiao. pcDNA3 and pEGFP-C1 plasmids were purchased from Clontech (Palo Alto, Calif.). pRc-hA3AR, consisting of the CMV promoter driving the expression of the human A3 adenosine receptor cDNA and pCMV-β-Gal, consisting of the CMV promoter driving the bacterial β-Galactosidase gene, were constructed in our lab and verified by DNA sequencing

Cell culture: MCF-7, ZR75 and T47D cells were originally from American Type Culture Collection (ATCC) and cultured in Dulbecco's Modified Eagle's Medium (DMEM, Invitrogen, Carlsbad, Calif.) supplemented with 10% fetal bovine serum (FBS), 5 U/ml penicillin, 5 μg/ml streptomycin, and 2 mM L-glutamine (All from Invitrogen, Carlsbad, Calif.). Hs578t cells were cultured in the above medium supplemented with 0.01 mg/ml insulin (Sigma, St. Louis, Mo.). When indicated, MCF-7 cells were cultured in DMEM medium free of phenol red (Invitrogen, Carlsbad, Calif.) with charcoal stripped serum (Hyclone, Logan, Utah) for 3 days before being treated with drugs.

Anchorage-independent growth (soft agar) assay: Soft agar assay was performed as described (40) with the following modifications. Ligands were added into the bottom and top agar before plating into 6-well plates. Cells were treated with trypsin (Invitrogen, Carlsbad, Calif.) for 5 minutes in a 37° C. incubator and pipetted several times so that most cells were in single cell forms. Cells were counted with a hemacytometer (Hausser Scientific/VWR, So. Plainfield, N.J.), and 10,000 cells were mixed with top agar and plated into each well. After the top agar had solidified, two ml of medium containing the same treatment was added on top of the agar. This covering medium was changed every two days during culture. After two weeks of culture, each well was counted for the number of colonies formed on an Olympus IX70 microscope under 40× optical amplification. A cell colony was defined as any cluster of cells that contain more than 3 cells. The average of counts from 3 random optical fields for each well was taken as the colony number and analyzed. Each treatment was performed in triplicates. The averages and standard deviations shown in the figures were calculated based on triplicate experiments.

Anchorage-dependent growth assay: Cells were plated into 6-well plates and grown overnight before treatments. The seeding concentration of MCF-7 cells was 2×10⁵/well, which was determined during preliminary experiments as not allowing the cells to reach confluency within 3 days. Cells were treated either with vehicle or ligands, as indicated. Cells were detached by incubation with trypsin (Invitrogen, Carlsbad, Calif.) and counted with a hemacytometer before or after treatment.

Western Blot Analysis: Cells were washed three times in cold 1×PBS, and collected by scraping on ice. Western blot analysis was performed as we described before with the chemiluminescence method (41). Antibodies used in this study were: ERα (NeoMarkers, Ab-15), Cyclin A (Santa Cruz Biotech, H-432), Cyclin B1 (Santa Cruz Biotech, H-433), Cyclin E (Upstate Biotech, HE-12), p27 (Santa Cruz Biotech, F-8). ERα antibody was used at 1:100 dilutions. Cyclin A, cyclin B1 and p27 antibodies were used at 1:500 dilutions. Cyclin E antibody was used at 1:1000 dilutions.

Flow Cytometry and Apoptosis Analysis: Cells were detached from tissue culture plates by trypsin treatment. Cells were collected by centrifugation at 1200 g for 5 minutes and washed once with PBS. Staining of cells with propidium iodide and analysis on a flow cytometer (FACScan, Becton Dickinson, Research Triangle Park, N.C.) was performed as described before (42). Data were analyzed with CellQuest software (Becton Dickinson, Research Triangle Park, N.C.). The percentage of cells appearing with a ploidy level smaller than a diploid content was calculated as an estimate of cells undergoing apoptosis.

Transfections and Reporter Gene Assay: Transient transfection was performed using FuGene6 (Roche, Indianapolis, Ind.) transfection reagent according to manufacturer's protocol. Circular reporter plasmid pERE-Tk-Luc at 10 μg and 10 μg of pCMV-β-Gal (as a measure of efficiency of trasnfection) were transfected with 50 μl of FuGene6. Cells were split into 6 well plates 12 hours after transfection, and incubated overnight in fresh medium. Cells were treated with vehicle or 100 μM IB-MECA for 12 hours before harvesting. Luciferase and β-galactosidase activities were measured as described before (43, 44).

Stable transfection was performed with similar procedures as transient transfection, except that the plasmid pRc-A3AR was linearized with PvuI, and purified by phenol/chloroform extraction and ethanol precipitation. Transfected MCF-7 cells were selected with 500 μg/ml of Geneticin (Invitrogen, Carlsbad, Calif.), until Geneticin treated control cells all died. This pool of stably transfected cells were either used in experiments, or subjected to single clone selection with limited dilution as described before (43). Briefly, cells were diluted into a concentration of 2.5 cells per ml, and added into 96 well plates at 200 μl/well. Clones of cells grown up were analyzed for their A3AR expression using RT-PCR.

Total RNA preparation and Reverse Transcription Polymerase Chain Reaction (RT-PCR): Total RNA from MCF-7 cells was prepared with Trizol (Invitrogen, Carlsbad, Calif.) as described before (41). For reverse transcription, 2 μg of RNA were used in a 20 μg reaction with random primers and M-MLV reverse transcriptase (Invitrogen, Carlsbad, Calif.), following the manufacturer's protocol. To control for possible contamination from genomic DNA in subsequent PCR reactions, control reverse transcription reactions were carried out under identical conditions, only without reverse transcriptase. After reverse transcription, 5% of the product was used in each PCR reaction. For the experiments analyzing A3 adenosine receptor (A3AR) expression, 27 cycles were used in the PCR reactions. Specific primers were designed for human A3AR, which match to two separate exons, according to genomic sequences (from GenBank). Sequences for the sense and antisense A3AR primers are: 5′tccatcatgtccttgctg3′ (SEQ ID NO: 1) and 5′gcacatgacaaccaggg3′ (SEQ ID NO.: 2). In the experiments analyzing estrogen receptor a (ERα) mRNA, semi-quantitative PCR reactions were carried out with 23 cycles for ERα primers and 19 cycles for GAPDH primers (used as a control). The cycle numbers were tested in previous experiments not to produce saturation effects. The sense and antisense primer sequences for estrogen receptor a are: 5′gatccaagggaacgagctgg3′ (SEQ ID NO.: 3) and 5′tgggctcgttctccaggtag3′ (SEQ ID NO.: 4). The sense and antisense primer sequences for GAPDH are: 5′tcaccatcttccaggag3′ (SEQ ID NO.: 5) and 5′gcttcaccaccttcttg3′ (SEQ ID NO.: 6).

Thymidine Incorporation Assay. Thymidine incorporation assays were performed as described (Zhang, Y., Wang, Z., and Ravid, K. The cell cycle in polyploid megakaryocytes is associated with reduced activity of cyclin B1-dependent cdc2 kinase. J Biol Chem, 271: 4266-4272, 1996) with modifications. Rat bone marrow cells were cultured in 25 cm² flasks at a concentration of 20×10⁶ cells per 2 ml. After drug treatment for 24 hours, cells were incubated with ³H-thymidine at a final concentration of 3 μCi/ml for 8 hours. For MCF-7 cells, cells were cultured in 6-well plates and incubated with ³H-thymidine for 2 hours after drug treatment. Cells were divided into two portions, sixty percent of which were processed as described (Id.) to obtain tritium counts. The rest of the cells were lysed with Western blotting lysis buffer and protein concentrations were determined by Bio-Rad protein assay reagent (Bio-Rad, Hercules, Calif.). Tritium counts were normalized with corresponding protein concentrations to account for cell number variations.

Messenger RNA Half-life Determination. MCF-7 cells were pretreated with vehicle (DMSO) or 100 μM IB-MECA for 6 hours, followed by addition of 80 μM of DRB (5,6-dichlorobenzimidazole riboside) or 50 μM of actinomycin D. Cells were harvested either before addition of transcription inhibitor (0 hour) or after different time periods. Total RNA was prepared and ERα content was assayed by RT-PCR analyses as described in the methods for RT-PCR. To control for the amount of RNA used in reverse transcription reactions, 2 μg each of total RNA were resolved on a denaturing agarose gel as described before (Cataldo, L. M., Zhang, Y., Lu, J., and Ravid, K. Rat NAP1: cDNA cloning and upregulation by Mp1 ligand. Gene, 226: 355-364, 1999) and stained with ethidium bromide.

Results

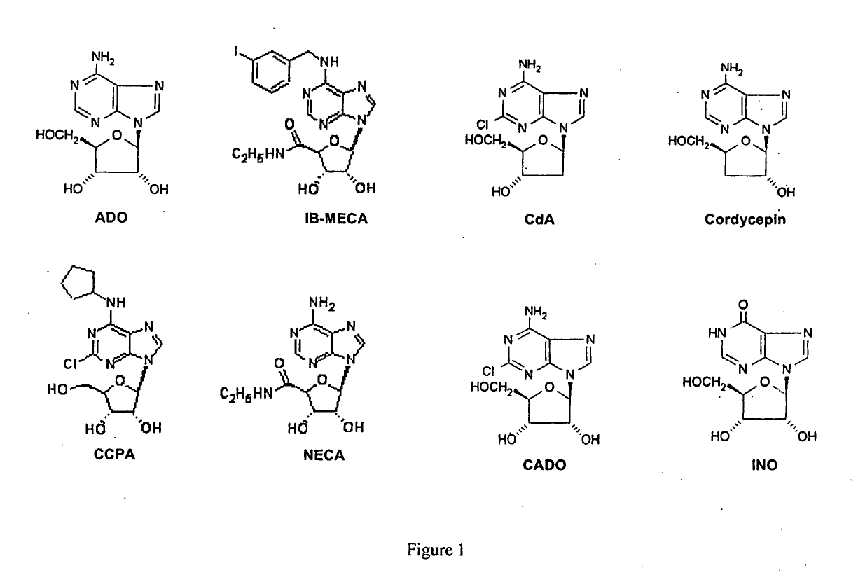

Adenosine or IB-MECA inhibits anchorage-independent growth of MCF-7 cells. It has been reported that skeletal muscle-conditioned medium, with adenosine as an active component, can inhibit anchorage-dependent growth of MCF-7 breast cancer cells, as measured by thymidine incorporation (15). We examined whether adenosine can also inhibit the anchorage-independent growth of MCF-7 cells, a hallmark of tumorogenesis, and if this effect was mimicked by adenosine analogs. Adenosine was added into soft agar cultures at different concentrations, and colonies formed were counted after two weeks of culturing. As shown in FIG. 1A, adenosine displayed a dose-dependent inhibition of colony formation. At 1 mM, adenosine inhibited approximately 50% of the colony-forming ability of MCF-7 cells. No effect was observed when inosine was used instead of adenosine (not shown).

Such a high concentration of adenosine can hardly be achieved during normal physiological processes. Since adenosine exerts many of its effects through the activation of adenosine receptors and many adenosine receptor agonists have a higher stability than adenosine, we asked whether agonists for the four types of adenosine receptors could inhibit anchorage-independent growth of MCF-7 cells. CCPA (A1AR agonist), NECA (A2AR agonist), CGS21680 (A2aAR agonist), and IB-MECA (A3AR agonist) were used at different concentrations. At much higher concentrations than their binding affinities, CCPA, NECA, and CGS21680 did not inhibit the anchorage-independent growth of MCF-7 cells (FIG. 1B through 1D). IB-MECA, on the other hand, at concentrations from 10 to 100 μM, showed a dose-dependent inhibition of MCF-7 cell colony formation (FIG. 1E).

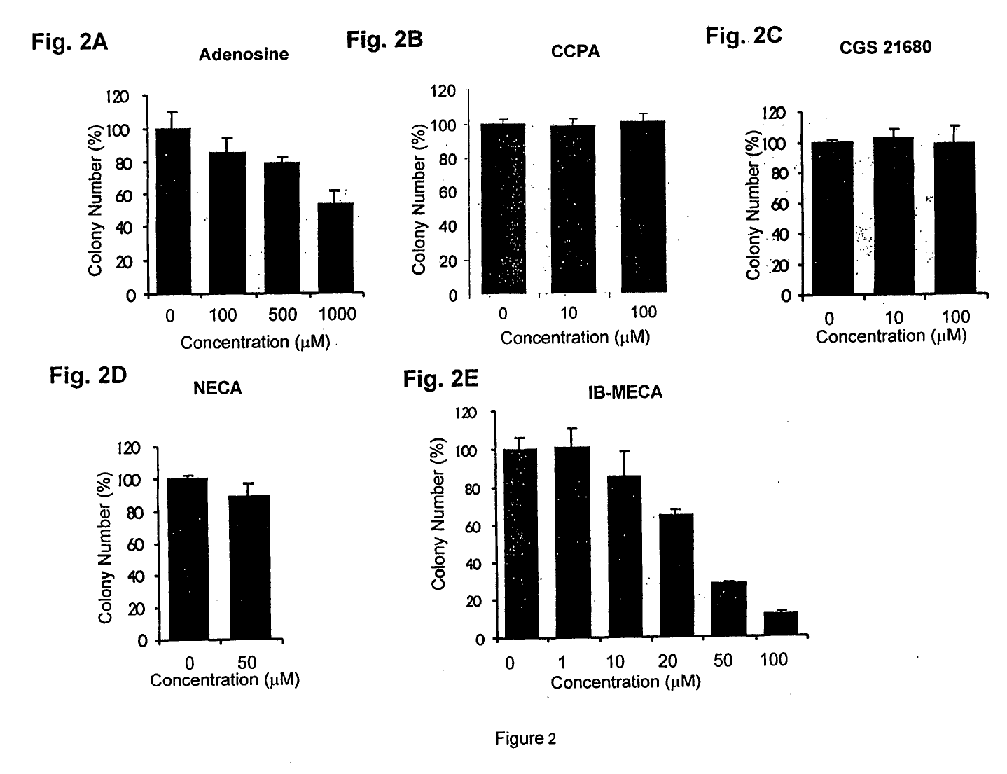

Effects of IB-MECA on anchorage-independent growth, anchorage-dependent growth and apoptosis of different breast cancer cell lines: We tested the effect of IB-MECA on several human breast cancer cell lines, including ZR-75, T47D (ERα positive), Hs578T (ERα negative) and HeLa (human cervix adenocarcinoma cell line). All breast cancer cell lines tested showed a dramatic decrease in colony formation, while HeLa cells only exhibited a mild response to this agonist (FIG. 2A), suggesting that inhibition of anchorage-independent growth by IB-MECA is closely related to the origin of cancer.

The effect of IB-MECA on anchorage-dependent growth was also examined in these breast cancer cell lines. After three days of treatment in culture, trypan blue negative cells were counted and compared to the cell counts on day 0 (before treatment). Inhibition of anchorage-dependent growth Was observed with all four breast cancer cell lines tested, namely MCF-7, ZR-75, T47D, and Hs578T. The cell counts after three days of treatment were all lower than those at day 0. Noticeably, however, cell counts of MCF-7 and ZR-75 cells decreased only mildly while T47D and Hs578T cells were affected more severely (FIG. 2B).

Some studies involving examination of mechanisms of IB-MECA effects on growth of a variety of transformed cells concluded that increased apoptosis is involved (13, 14). We examined whether the fraction of apoptotic cells was increased in IB-MECA-treated breast cancer cells. We have elected a quantitative approach to follow apoptotic cells. To this end, cells were stained with propidium iodide after ligand treatment, and subjected to flow cytometry analyses. The fraction of events with fluorescence intensity less than a diploid DNA content would indicate the relative population of apoptotic cells. As shown in FIG. 2C, T47D and Hs578T cells treated with IB-MECA underwent substantial apoptosis compared to the vehicle-treated samples, while MCF-7 and T47D cells displayed a non significant change in apoptotic events.

These results indicated that IB-MECA can induce two types of signaling in breast cancer cells. One involves growth inhibition and another induces apoptosis. IB-MECA-induced growth arrest in ER positive breast cancer cells, however, has never been reported, and this study will focus on elucidating the mechanisms of such an effect.

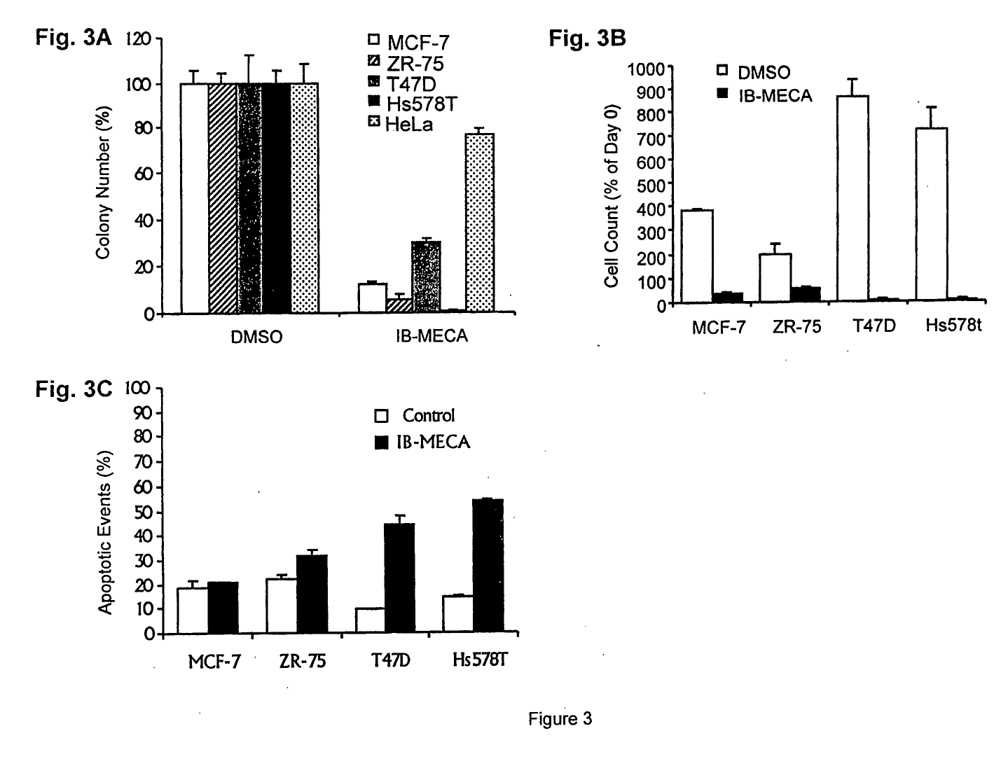

IB-MECA inhibits anchorage-dependent proliferation of MCF-7 cells: Since IB-MECA inhibited the anchorage-independent proliferation of MCF-7 cells on both colony numbers and sizes we further tested this chemical on the anchorage-dependent proliferation of these cells. The numbers of trypan-blue negative cells were followed after MCF-7 cells were treated with IB-MECA. Although vehicle treated cells showed an exponential increase in cell count, cells treated with IB-MECA did not show much change in the number of viable cells, even after 3 days of drug treatment (FIG. 3A). Our data indicated that IB-MECA was able to rapidly inhibit anchorage-dependent proliferation of MCF-7 cells.

We further tested this inhibition by analyzing DNA synthesis through thymidine incorporation. Because many chemicals that inhibit cancer cell proliferation have undesirable side-effects on bone marrow cells, we also tested the effect of IB-MECA on a primary rat bone marrow culture through thymidine incorporation. Since bone marrow cells have much lower rates of proliferation after long periods in culture (not shown) and the effect of IB-MECA on MCF-7 cells could be observed after 1 day, we treated the cells for 24 hours before incubating them with thymidine. IB-MECA and 2-chloro-2′-deoxyadenosine (2CdA, a drug used in chemotherapy) decreased thymidine incorporation in MCF-7 cells to 28% and 43% respectively (FIG. 3B). In contrast, IB-MECA at 100 μM had a milder effect on thymidine incorporation in primary bone marrow cells (reduced to 68%), compared to the effect of 2CdA (reduced to 32%) (FIG. 3C). Interestingly, in vivo application of IB-MECA had no inhibitory effect on blood cell counts, probably due to cytokine influences (Fishman, P., Bar-Yehuda, S., Madi, L., and Cohn, I. A3 adenosine receptor as a target for cancer therapy. Anticancer Drugs, 13: 437-443, 2002).

IB-MECA arrests MCF-7 cells at G1 or G1/S phase of the cell cycle. We then explored which point of the cell cycle was blocked by treatment of IB-MECA. Flow cytometry analysis of MCF-7 cells treated with IB-MECA, compared to vehicle-treated cells, showed that there was a decrease of S-phase population from 25% to 12% (FIG. 3B). The peak with diploid DNA content increased from 50% to 64% after IB-MECA treatment. There was also a minute decrease in the population of tetraploid DNA content from 24% to 23%. These results suggested that IB-MECA has a primary effect on the G1/S cell cycle transition.

To further analyze the cell cycle arrest, Western blot analyses were carried out with antibodies against cyclins and Cdk inhibitors. As shown in FIG. 3C, cyclin A, and B1 were downregulated in MCF-7 cells. Consistent with the previous growth inhibition data, HeLa cells showed no significant change in cyclin levels (data not shown). The decrease in cyclins A and B1 was accompanied by a sharp increase in the cdk inhibitor p27. Cyclin E levels were elevated upon ligand treatment, as might be expected from cell cycle arrest at G1 phase. These data confirmed that the cell cycle inhibition was primarily at G1/S. Interestingly, treating MCF-7 cells with different concentrations of IB-MECA showed decreases in cyclins A and B with a similar dosage response as the decrease in anchorage-independent growth (FIG. 3D).

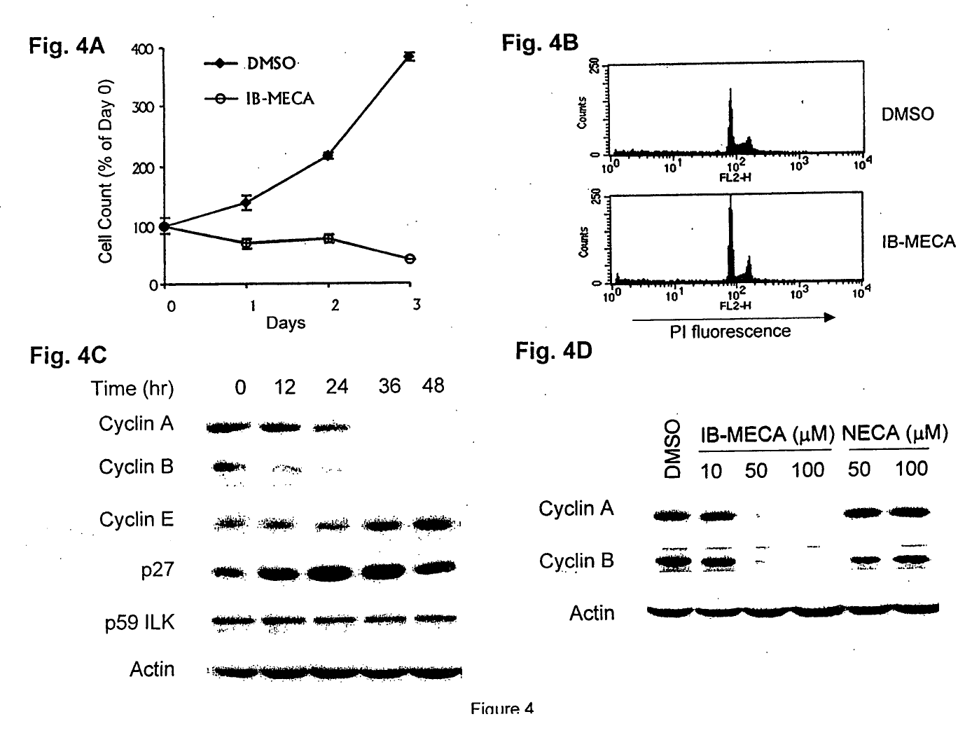

Overexpression of A3AR does not increase the sensitivity of MCF-7 cells towards IB-MECA treatment: IB-MECA is an A3AR selective agonist. Cell growth inhibition by IB-MECA in several transformed cell lines has been attributed to A3AR activation (31-33). The affinity of IB-MECA for A3 adenosine receptor was reported to be in the nanomolar range (34). However, the cell growth inhibitory effect we report here could only been observed at concentrations higher than about 10 μM. There might two possible reasons that could explain why the concentration needed for growth inhibition is much greater than the binding affinity. One possibility is that the growth inhibition is not mediated through the A3AR. The second possibility is that MCF-7 cells have low abundance or/and low affinity A3AR, so that only a high concentration of IB-MECA can activate a relevant downstream signaling. Indeed, MCF-7 only had a very low level of A3AR expression (FIG. 4A), as endogenous A3AR mRNA could barely be detected after 33 cycles of RT-PCR reactions (data not shown). If the growth inhibition by IB-MECA was mediated by low level expression of A3AR in MCF-7 cells, overexpression of the human A3AR would increase the sensitivity of cells upon IB-MECA treatment. To explore this possibility, MCF-7 cells were stably transfected with human A3AR cDNA. Expression of A3AR in a stable transfection pool could be strongly detected with 27 cycles of RT-PCR reactions (FIG. 4A), and was stronger than the expression level in the brain, where A3AR is abundantly expressed (data not shown). The percentage of cells in the transfection pool that contain the transgene was estimated by analyzing single clones selected from the pool of cells. 16 out of 17 clones showed strong increased expression of A3AR (data not shown), verifying that majority of the cells within the transfection pool overexpressed A3AR. The pool of stably transfected cells were compared to normal MCF-7 cells in soft agar assays. FIG. 4B shows that the two types of cells have almost identical dosage response to IB-MECA. Increased expression did not lower the concentration of IB-MECA needed to induce growth suppression. In accordance, the selective A3AR antagonist MRS1191 used at a concentration of up to 1 μM (greater than its Ki) did not abolish IB-MECA inhibitory effect on growth of MCF-7 cells (not shown). Thus, we concluded that the growth inhibition by IB-MECA is not mediated through A3AR.

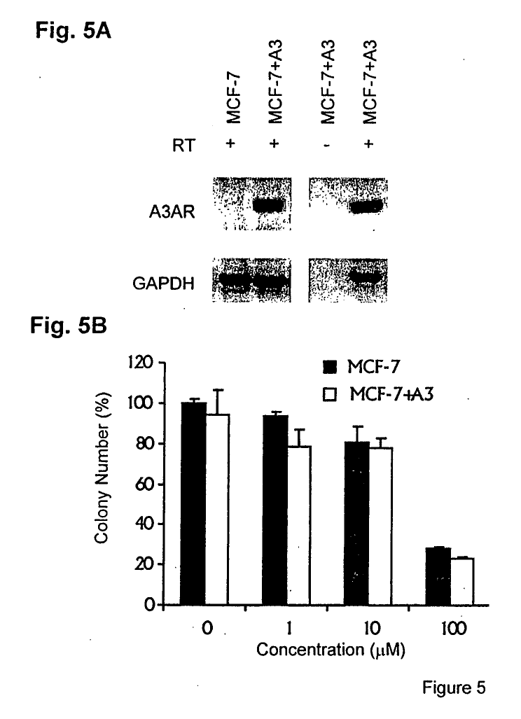

IB-MECA treatment decreases estrogen receptor a in MCF-7 cells: The data described above indicated that IB-MECA inhibits cell cycle progression primarily at the G1/S transition. Since estrogen receptor activation is known to promote cell cycle progression though G1/S and enhance both anchorage-dependent and anchorage-independent growth of breast cancer cells, we asked whether ERα is a primary target of IB-MECA. To this end, the expression of endogenous ERα mRNA was analyzed with semi-quantitative RT-PCR in MCF-7 cells treated with IB-MECA. A decrease in the expression of ERα could be consistently detected at 6 hours post IB-MECA treatment, compared to GAPDH expression (FIG. 5A). A dramatic decrease of ERα could be detected after 12 hours and 24 hours of treatment. This result shows that IB-MECA treatment can either reduce transcription driven by the ERα promoter gene or affect the stability of ERα mRNA. Detailed mechanism of this downregulation is under investigation.

As a consequence of a downregulation of ERα mRNA, ERα protein should also show a time-dependent decrease in IB-MECA-treated cells. Indeed, Western blot analyses indicated that ERα protein in MCF-7 cells decreased after IB-MECA treatment, and this decrease occurred before the reduction in cyclin levels (FIG. 5B), suggesting that ERα downregulation could be the reason for cell cycle inhibition. Reduction of ERα protein levels was also observed in ZR-75 and T47D cells treated with IB-MECA (not shown), suggesting that the impact of IB-MECA on this protein is common to ERα-positive breast cancer cell lines. Using different concentrations of IB-MECA, ERα showed a dosage response very similar to the one of cyclins and the growth inhibition (FIG. 5C). In contrast, no significant change of ERα was detected in cells treated with NECA (FIG. 5C). These results further suggested that the decrease of ERα might be responsible for the growth inhibition effect of IB-MECA. Different batches of IB-MECA and cell culture medium might have had an impact on the rate of ERα downregulation. However, this decrease could always be detected, between 4 and 8 hours post ligand treatment, under both normal culturing condition and with phenol-red free cell culture medium and charcoal stripped serum, as well as with two different batches of IB-MECA (data not shown).

Since activators of estrogen receptors, such as 17-β-estrodiol, can reduce ERα level by regulating ERα protein stability, a decrease of protein may not correlate with a decrease of ERα activity (Wijayaratne, A. L. and McDonnell, D. P. The human estrogen receptor-alpha is a ubiquitinated protein whose stability is affected differentially by agonists, antagonists, and selective estrogen receptor modulators. J Biol Chem, 276: 35684-35692, 2001; Borras, M., Laios, I., el Khissiin, A., Seo, H. S., Lempereur, F., Legros, N., and Leclercq, G. Estrogenic and antiestrogenic regulation of the half-life of covalently labeled estrogen receptor in MCF-7 breast cancer cells. J Steroid Biochem Mol Biol, 57: 203-213, 1996). We next examined the transcriptional activity of endogenous ERα after IB-MECA treatment, using a reporter construct containing estrogen responsive elements (EREs) in the promoter. Shown in FIG. 5D, after 12 hours of IB-MECA treatment, the ERE promoter activity dropped by more than 5 fold. In contrast, the non-tissue specific CMV promoter did not show any decrease in activity. These results indicated that IB-MECA downregulated ERα and subsequently caused a reduced activity of this transcription factor.

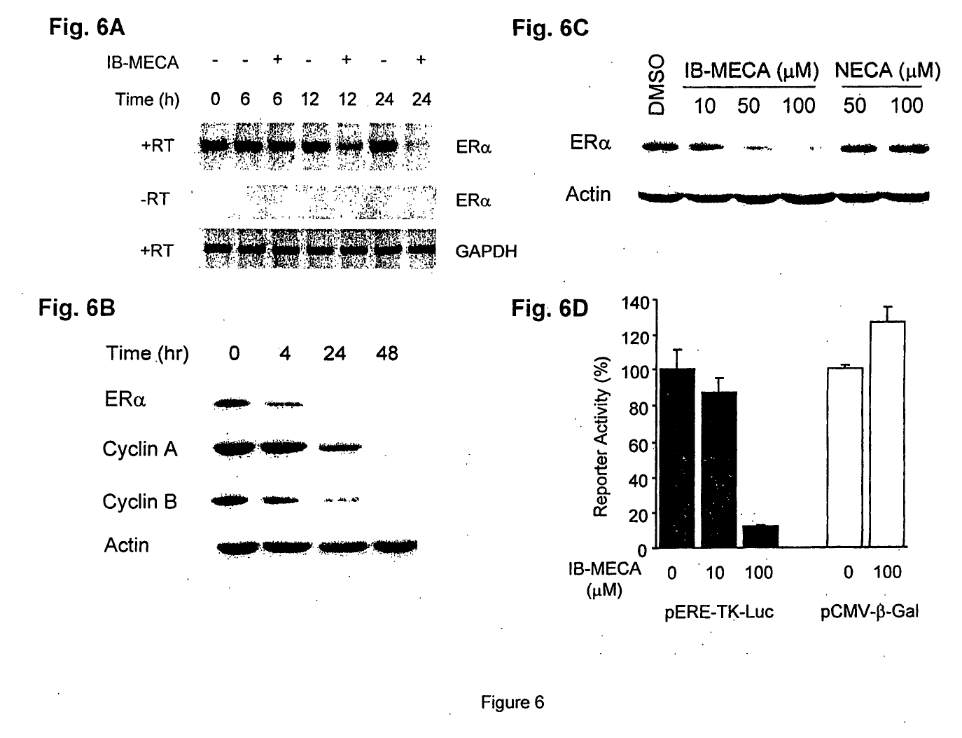

Overexpression of ERα can reverse the growth inhibition induced by IB-MECA treatment: Results from the above experiments indicate that the growth inhibition of IB-MECA is mediated through a decrease in ERα. To verify this hypothesis, we took advantage of the fact that the CMV promoter is not affected much by IB-MECA treatment, and hence transfected cells with ERα cDNA under the control of the CMV promoter. This would provide the cells with abundance of ERα. Indeed, ERα levels were high in the transfected cells, even after IB-MECA treatment (FIG. 6A).

If the inhibition of IB-MECA was mediated through a decrease in ERα, we would expect to see a moderate or no effect of IB-MECA on growth of ERα overexpressing cells. Since only cells transfected with ERα may exhibit any resistance, transfection efficiency will be key in determining the potential increase in cell counts as compared to control non-transfected cells. Stable transfection of ERα was attempted twice without success, suggesting a potential long term harmful effect of high levels of ERα in MCF-7 cells. Instead, we overexpressed ERα by transient transfection, and a transfection efficiency of approximately 40% was determined by counting green cells from a parallel transfection with a CMV-driven green fluorescence protein construct (pEGFP-C1).

When ERα was overexpressed in MCF-7 cells, IB-MECA treatment resulted in a moderate effect on growth, as compared to a larger effect in control cells (FIG. 6B). It is reasonable to assume that IB-MECA effect on growth of the transfected pool of cells was not eliminated because of only approximately 40% of the cells overexpressed the transgene.

Following methods of assays described above, we analyzed the ability of other adenosine analogs and of the nucleosides adenosine and inosine in respect to the ability to inhibit the growth of MCF-7 cells and affect ERα protein levels. Table 1 summarizes the data obtained. Inosine as well as the A1AR selective analog, CCPA, or the A2-type selective analog NECA had no significant effect on cell growth. We also examined adenosine analogs, which are not selective for adenosine receptors and have been described as inhibitors of cancer cells. For example, 2-chloro-deoxyadenosine was used in trials for treating chronic lymphocytic leukemia (45), or infantile myofibromatosis (46), and 3′-deoxyadenosine was shown to inhibit leukemia cells (47). In our studies, 2-chloror-deoxyadenosine significantly inhibited the growth of MCF-7 human breast cancer cells. In contrast to IB-MECA, however, it was as effective at 1 μM (not shown) as at 100 μM and it did not have a prominent effect on ERα levels, but induced cellular apoptosis. 2-chloro-adenosine>3′-deoxyadenosine significantly inhibited cell growth and ERα levels, without inducing apoptosis. These compounds, as IB-MECA were only effective when used at a 10-100 micromolar range. They are likely, however, to act on a different mechanism than IB-MECA because they affected the cell cycle at a different phase, i.e., not at G1/S as IB-MECA did. Hence, their reducing effect on ER (which is attenuated as compared to the effect of IB-MECA) might be a result of a primary effect on cell cycle arrest. These data show that IB-MECA, 2-chloro-adenosine as well as 3′-deoxyadenosine can be used in vivo for inhibition of breast cancer. 3′-deoxyadenosine (Cordycepin) was used before in Clinical Trials to inhibit specific blood cell cancers.

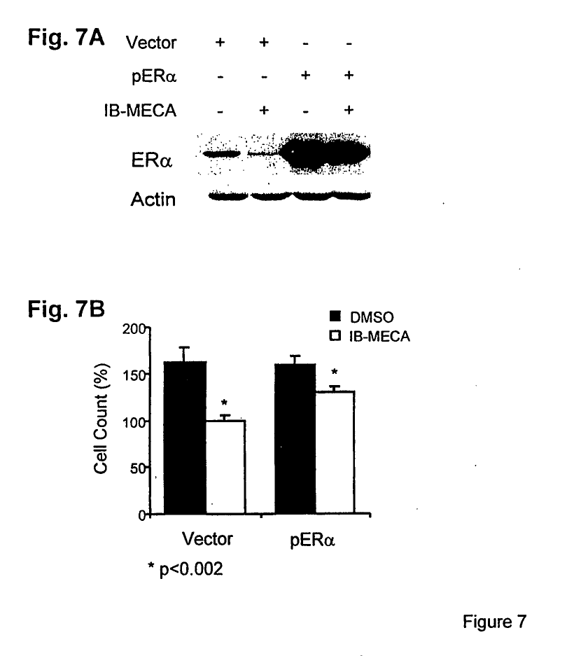

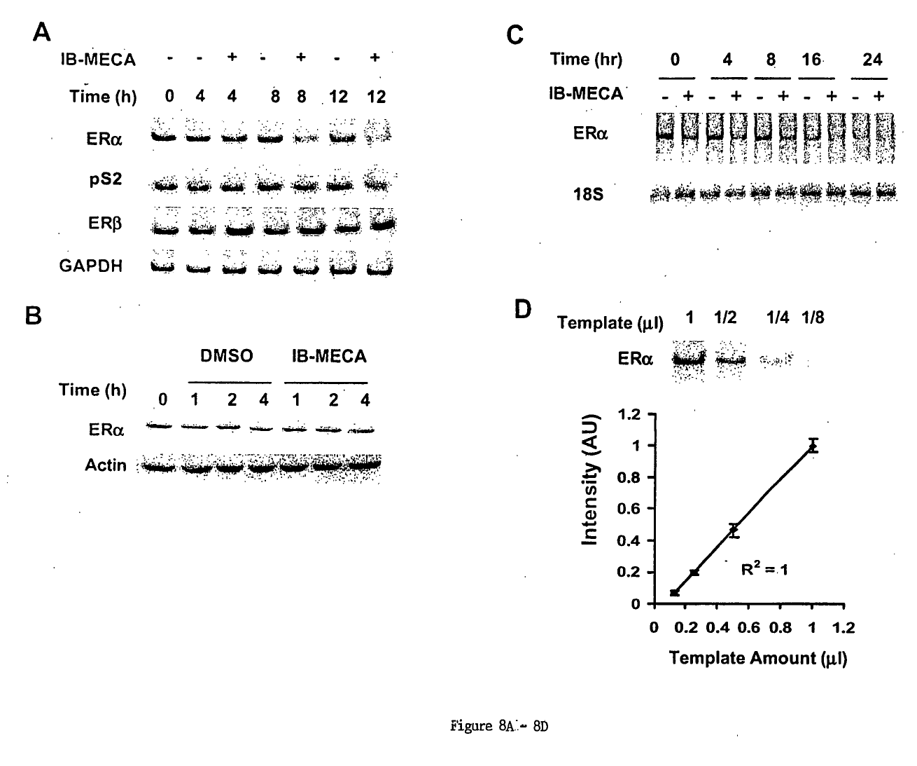

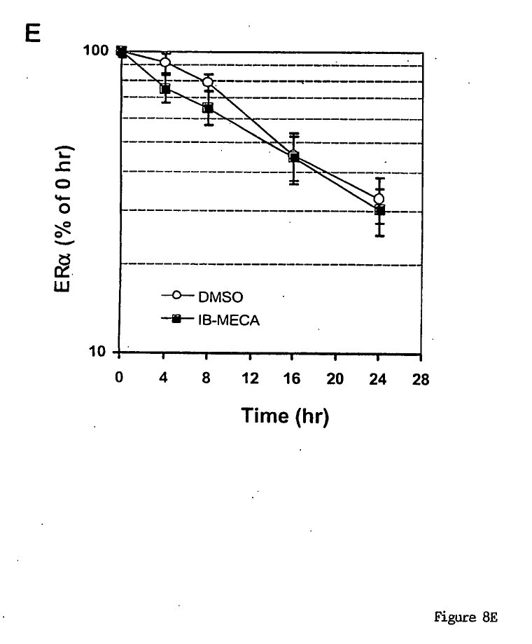

IB-MECA-induced downregulation of ERα is likely die to decreased transcription from the estrogen receptor a gene. Studies on ERα regulation have revealed that this gene is regulated at the levels of transcription (McPherson, L. A., Baichwal, V. R., and Weigel, R. J. Identification of ERF-1 as a member of the AP2 transcription factor family. Proc Natl Acad Sci USA, 94: 4342-4347, 1997), mRNA stability (Saceda, M., Lindsey, R. K., Solomon, H., Angeloni, S. V., and Martin, M. B. Estradiol regulates estrogen receptor mRNA stability. J Steroid Biochem Mol Biol, 66: 113-120, 1998; Ing, N. H. and Ott, T. L. Estradiol up-regulates estrogen receptor-alpha messenger ribonucleic acid in sheep endometrium by increasing its stability. Biol Reprod, 60: 134-139, 1999; Kenealy, M. R., Flouriot, G., Sonntag-Buck, V., Dandekar, T., Brand, H., and Gamnon, F. The 3′-untranslated region of the human estrogen receptor alpha gene mediates rapid messenger ribonucleic acid turnover. Endocrinology, 141: 2805-2813, 2000), and protein degradation (Wijayaratne, A. L. and McDonnell, D. P. The human estrogen receptor-alpha is a ubiquitinated protein whose stability is affected differentially by agonists, antagonists, and selective estrogen receptor modulators. J Biol Chem, 276: 35684-35692, 2001; Borras, M., Laios, I., el Khissiin, A., Seo, H. S., Lempereur, F., Legros, N., and Leclercq, G. Estrogenic and antiestrogenic regulation of the half-life of covalently labeled estrogen receptor in MCF-7 breast cancer cells. J Steroid Biochem Mol Biol, 57: 203-213, 1996). We investigated the downregulation induced by IB-MECA by first examining the abundance of ERα mRNA. For comparison between protein levels and mRNA levels, materials from the same samples as in FIG. 6B were used for total RNA preparation. As shown in FIG. 7A, IB-MECA strongly downregulated ERα mRNA in a fast and time-dependent manner, with the first sign of decrease after 4 hours of IB-MECA treatment. Following ERα downregulation, the mRNA level of pS2, an endogenous estrogen-responsive gene (55), was also reduced by IB-MECA. The downregulation of pS2 could be observed after 8 to 12 hours (FIG. 7A), and to a stronger degree after 24 hours (not shown). In contrast, the message level of another estrogen binding protein, estrogen receptor β (56), was not significantly reduced (FIG. 7A). This cDNA was amplified with primers corresponding to the first two exons of the estrogen receptor β gene. ERα mRNA downregulation precedes that of ERα protein (FIGS. 6B and 8A), suggesting that the primary effect of IB-MECA is on ERα mRNA. Indeed, we did not notice any significant change in ERα protein degradation upon IB-MECA treatment, when protein synthesis was inhibited by cycloheximide (FIG. 8B).

To differentiate whether the effect was on ERα gene transcription or mRNA stability, we examined the half-life of ERα message in vehicle- or IB-MECA-treated cells. MCF-7 cells were pretreated with vehicle or IB-MECA for 6 hours before adding the transcription inhibitor 5,6-dichlorobenzimidazole riboside (DRB) which causes premature transcription termination. Consistent with FIG. 8A, ERα mRNA was decreased after 6 hours of IB-MECA pretreatment, as revealed by semi-quantitative RT-PCR (the 0 hour time point, FIG. 8C). The PCR reactions were carried out under conditions that allow linear amplification and quantitation (FIG. 8D). The half-life of ERα measured under the experimental conditions may be longer than the real half-life in the cells, since the used transcription inhibitor may not shut down transcription immediately. Nevertheless, comparing vehicle- and IB-MECA-treated cells would indicate whether there is an impressive difference in ERα half-lives. The mRNA half-life in IB-MECA-treated cells was similar to that in vehicle-treated cells (FIGS. 8C and 8E), and the difference could not account for the observed reduction in mRNA level. Inhibiting transcription with another transcription inhibitor, actinomycin D, showed similar results (not shown). Thus, we concluded that the effect of IB-MECA on ERα was most likely on the transcription of the gene. It should be pointed out that nuclear run-on assays were attempted, as we described before (Wang, Z., Zhang, Y., Lu, J., Sun, S., and Ravid, K. Mp1 ligand enhances the transcription of the cyclin D3 gene: a potential role for Sp1 transcription factor. Blood, 93: 4208-4221, 1999), but ERα de novo transcription in control cells was below our detection limit.

Discussion

While adenosine and chemically-synthesized adenosine receptor agonists have been reported to inhibit cancer cell proliferation, these inhibitory effects are through various mechanisms, and mainly via the activation of different adenosine receptors. In contrast, found that high concentrations of adenosine inhibited growth of cancers having anchorage-independent cells such as MCF-7 breast cancer cells. Among the agonists examined in our study, IB-MECA was shown to be a potent growth inhibitor of breast cancer cell lines, while the A₁AR agonist, CCPA, and the A₂AR agonists, CGS21680 and NECA, did not have a significant effect on MCF-7 cell proliferation. The breast cancer cells examined showed no detectible levels of A₃AR, and A₃AR overexpression in MCF-7 cells did not result in increased sensitivity upon IB-MECA treatment. In addition, an A₃AR antagonist did not abolish the effect of IB-MECA. This suggested that A₃AR is not a primary pathway through which the growth inhibition is mediated. Another A₃AR agonist, chloro-IB-MECA, was shown to induce apoptosis in two leukemia cell lines, through mechanisms not related to A₃AR signaling (Kim, S. G., Ravi, G., Hoffmann, C., Jung, Y. J., Kim, M., Chen, A., and Jacobson, K. A. p53-Independent induction of Fas and apoptosis in leukemic cells by an adenosine derivative, C1-IB-MECA. Biochem Pharmacol, 63: 871-880, 2002).

How IB-MECA triggers the effect on proliferation in the treated breast cancer cells is not clear. It is possible that IB-MECA at high concentrations binds other unidentified membrane receptors and triggers downstream signaling. Another possibility could be that the compound signals through direct interaction with intracellular targets, after being transported into the cell. Such intracellular mechanisms have been noticed for adenosine (Schrier, S. M., van Tilburg, E. W., van der Meulen, H., Ijzerman, A. P., Mulder, G. J., and Nagelkerke, J. F. Extracellular adenosine-induced apoptosis in mouse neuroblastoma cells: studies on involvement of adenosine receptors and adenosine uptake. Biochem Pharmacol, 61: 417-425, 2001) and an adenosine analog, 2-chloroadenosine (Barbieri, D., Abbracchio, M. P., Salvioli, S., Monti, D., Cossarizza, A., Ceruti, S., Brambilla, R., Cattabeni, F., Jacobson, K. A., and Franceschi, C. Apoptosis by 2-chloro-2′-deoxy-adenosine and 2-chloro-adenosine in human peripheral blood mononuclear cells. Neurochem Int, 32: 493-504, 1998), using the nucleoside transporter inhibitor dipyridamole. In our system, 10 μM dipyridamole did not prevent the growth inhibitory effect of IB-MECA, while at higher concentrations, dipyridamole had by itself an inhibitory effect on cell growth. It is possible, however, that the nucleoside uptake inhibitor can not fully block the transport of IB-MECA, since IB-MECA at higher concentrations might compete for the transporters or enter the cell by a nucleoside transporter-independent mechanism. In lack of radio-labeled IB-MECA, we were not able to determine the intracellular concentration of this ligand. The details of the mechanisms by which IB-MECA targets its effecter molecules are intriguing and await further exploration.

In search for mechanisms of action of IB-MECA on breast cancer cell growth, we focused on a known regulator of these cells, the estrogen receptor α. We showed that, in ERα-positive breast cancer cell lines MCF-7, ZR-75, and T47, IB-MECA downregulated ERα, suggesting that this effect is general in ERα-positive breast cancer cells. We also showed that reversing the downregulation of ERα significantly attenuated the growth inhibition induced by IB-MECA, indicating that ERα downregulation is one pathway responsible for the growth inhibition in ER-positive breast cancer cells. This, however, does not exclude the possibility that other pathways are also involved in IB-MECA-induced proliferation inhibition in these cells. We found that IB-MECA regulated ERα through a downregulation of mRNA and protein, and consequently ERα a transcriptional activity. The half-life of ERα message was not significantly altered when IB-MECA was present. This eliminates the possibility of regulation on message stability and points to a high likelihood of regulation through gene transcription. The ERα gene contains multiple promoters, some of which are as far as 150 kb upstream of the primary transcriptional start site (Kos, M., Reid, G., Denger, S., and Gannon, F. Minireview: genomic organization of the human ERalpha gene promoter region. Mol Endocrinol, 15: 2057-2063, 2001; Reid, G., Denger, S., Kos, M., and Gannon, F. Human estrogen receptor-alpha: regulation by synthesis, modification and degradation. Cell Mol Life Sci, 59: 821-831, 2002). Only a few transcription factors are known to regulate ERα gene expression (McPherson, L. A., Baichwal, V. R., and Weigel, R. J. Identification of ERF-1 as a member of the AP2 transcription factor family. Proc Natl Acad Sci USA, 94: 4342-4347, 1997; Cohn, C. S., Sullivan, J. A., Kiefer, T., and Hill, S. M. Identification of an enhancer element in the estrogen receptor upstream region: implications for regulation of ER transcription in breast cancer. Mol Cell Endocrinol, 158: 25-36, 1999), including AP2γ. Further experiments are needed to elucidate the detailed mechanism of ERα gene downregulation by IB-MECA. The mechanism by which IB-MECA downregulates ERα is different from the one found for selective estrogen receptor downregulators, such as ICI 182,780 (also known as fulvestrant and Faslodex). ICI 182,780 reduces ERα level through increased ERα protein turnover (Wakeling, A. E., Dukes, M., and Bowler, J. A potent specific pure antiestrogen with clinical potential. Cancer Res, 51: 3867-3873, 1991; Pink, J. J. and Jordan, V. C. Models of estrogen receptor regulation by estrogens and antiestrogens in breast cancer cell lines. Cancer Res, 56: 2321-2330, 1996; Fan, M., Bigsby, R. M., and Nephew, K. P. The NEDD8 pathway is required for proteasome-mediated degradation of human estrogen receptor (ER)-alpha and essential for the antiproliferative activity of ICI 182,780 in ERalpha-positive breast cancer cells. Mol Endocrinol, 17: 356-365, 2003), while IB-MECA downregulates ERα through an effect on gene expression. In this view, IB-MECA and similar compounds may be efficacious in the treatment of breast cancers that are resistant to or have acquired resistance (Lykkesfeldt, A. E., Larsen, S. S., and Briand, P. Human breast cancer cell lines resistant to pure anti-estrogens are sensitive to tamoxifen treatment. Int J Cancer, 61: 529-534, 1995) to the pure antiestrogen ICI 182,780, and thus might be important additions to the arsenal of endocrine therapies for human breast cancer.

We examined the effect of IB-MECA on several different breast cancer cell lines. IB-MECA inhibited the growth of MCF-7 and ZR-75 cells, and induced apoptosis in T47D and Hs578T cells. In T47D cells, IB-MECA treatment downregulated estrogen receptor a in a similar manner as in MCF-7 cells. It is known that T47D cells are estrogen-signaling-dependent; estrogen stimulates the proliferation of T47D cells, and inhibiting estrogen signaling results in an inhibition of proliferation (Jones, J. L., Daley, B. J., Enderson, B. L., Zhou, J. R., and Karlstad, M. D. Genistein inhibits tamoxifen effects on cell proliferation and cell cycle arrest in T47D breast cancer cells. Am Surg, 68: 575-577; discussion 577-578, 2002; Fontana, J. A. Interaction of retinoids and tamoxifen on the inhibition of human mammary carcinoma cell proliferation. Exp Cell Biol, 55: 136-144, 1987; Dardes, R. C., O'Regan, R. M., Gajdos, C., Robinson, S. P., Bentrem, D., De Los Reyes, A., and Jordan, V. C. Effects of a new clinically relevant antiestrogen (GW5638) related to tamoxifen on breast and endometrial cancer growth in vivo. Clin Cancer Res, 8: 1995-2001, 2002; Cavailles, V., Gompel, A., Portois, M. C., Thenot, S., Mabon, N., and Vignon, F. Comparative activity of pulsed or continuous estradiol exposure on gene expression and proliferation of normal and tumoral human breast cells. J Mol Endocrinol, 28: 165-175, 2002). Thus, it is possible that in T47D cells, two different pathways were induced by IB-MECA. One pathway involves ERα which is common in all ERα-positive cells and which would lead to proliferation inhibition. Another pathway, which is not activated in MCF-7 cells and ZR-75 cells, initiates apoptosis in T47D cells. In MCF-7 cells, IB-MECA does not induce or inhibit apoptosis. Apoptotic events can be initiated via a variety of signaling pathways and by activation of one or more related regulators (reviewed in Green, D. R. and Reed, J. C. Mitochondria and apoptosis. Science, 281: 1309-1312, 1998; Wajant, H. The Fas signaling pathway: more than a paradigm. Science, 296: 1635-1636, 2002; Vousden, K. H. p53: death star. Cell, 103: 691-694, 2000). We speculate that IB-MECA does not initiate apoptosis in MCF-7 cells because of its ability to activate certain anti-apoptotic molecules, such as Akt (reviewed in Franke, T. F., Kaplan, D. R., and Cantley, L. C. PI3K: downstream AKTion blocks apoptosis. Cell, 88: 435-437, 1997; Datta, S. R., Brunet, A., and Greenberg, M. E. Cellular survival: a play in three Akts. Genes Dev, 13: 2905-2927, 1999) so that the balance between its apoptotic and anti-apoptotic signals are maintained. Hence, the dominant effect of IB-MECA in MCF-7 cells is inhibition of ERα expression and proliferation. We found that IB-MECA induced Akt phosphorylation (at Ser 473) in MCF-7 cells (not shown), as also reported in rat basophilic leukemia 2H3 cells (Gao, Z., Li, B. S., Day, Y. J., and Linden, J. A3 adenosine receptor activation triggers phosphorylation of protein kinase B and protects rat basophilic leukemia 2H3 mast cells from apoptosis. Mol Pharmacol, 59: 76-82, 2001). This does not imply, however, that Akt is the only pathway by which IB-MECA might affect apoptosis in these cells. Further exploration is needed to reveal the detailed mechanisms by which apoptosis is induced by IB-MECA in some cell lines, but not in others.

In summary, we made the novel findings that IB-MECA potently inhibits ER positive cancer cell growth via downregulation of ERα, rather than through A3AR signaling. This shows that IB-MECA, and its functional derivatives can be used as a drug to treat patients with cancers expressing estrogen receptors, such as breast cancer. It may also be used in therapies that are aimed at regulating ERα levels. A variety of other adenosine analogs might be screened for inhibition of growth of breast cancer cells in vitro, using the tools we employed here.

TABLE 1
Effects of Different Adenosine Analogues on the
Growth of the Breast Cancer Cell Line MCF-7

| Compound¹ | Cell Growth² | Apoptosis³ | ERα Level⁴ | Cell Cycle Arrest⁵ |
|---|---|---|---|---|
| Control (Vehicle) | 1 | 1 | 1 | None |
| N⁶-(3- | −0.19 ± 0.06 | 1.12 ± 0.02 | 0.23 | G1/S |
| iodobenzyl)adenosine- | | | | (28, −51, −5) |
| 5′-N- | | | | |
| methyluronamide | | | | |
| (IB-MECA) | | | | |
| 2-chloro-deoxy- | 0.13 ± 0.01 | 1.80 ± 0.17 | 0.85 | S |
| adenosine (CdA) | | | | (−69, 165, 20) |
| 3′-deoxyadenosine | 0.24 ± 6 | 0.43 ± 0.07 | 0.58 | G2/M |
| (Cordycepin) | | | | (−26, 10, 62) |
| 2-chloro-N⁶- | 0.79 ± 0.16 | 0.49 ± 0.12 | 1.16 | G2/M |
| cyclopentyladenosine | | | | (3, −18, 11) |
| (CCPA) | | | | |
| 5′-(N- | 0.90 ± 0.16 | 0.99 ± 0.05 | 0.92 | Not Detectable |
| Ethylcarboxami- | | | | (0, 3, −3) |
| do)adenosine (NECA) | | | | |
| 2-chloro-adenosine | −0.02 ± 0.02 | 0.84 ± 0.6 | 0.41 | G2/M and S |
| (CADO) | | | | (−11, 11, 20) |
| Adenosine | 0.49 ± 0.04 | 1.06 ± 0.17 | 1.07 | G2/M |
| (ADO) | | | | (−25, −56, 97) |
| Inosine | 0.90 ± 0.12 | 1.02 ± 0.14 | ND | S |
| (INO) | | | | (−24, 43, 8) |

¹Compounds were used at 100 μM, except for adenosine (500 μM) and inosine (1 mM).
²Viable cell numbers were determined by staining with Trypan Blue and counting on a hematocytometer. Cells were counted after 2 days of treament and cell growth was calculated, normalizing to vehicle control (arbitrarily set to 1). A negative number means a decrease of cell number compared to cell count right before treatment. Data shown are averages ± standard deviations from three experiments.
³Data represent the number of apoptotic cells after 2 days of treatment compared to vehicle control, determined as described under Methods. In vehicle control, 10% to 20% of the cells were apoptotic and were arbitrarily set to 1. Data are averages ± standard deviations from three experiments.
⁴Cells were treated with indicated drugs for 12 hours and ERα protein level was measured by Western blot analyses, and quantitated using Kodak Digital Scientific 1D software. The ERα level of vehicle control was arbitrarily set to 1, and data represent the averages of two experiments.
⁵Cell cycle arrest was determined by flowcytometry analyses. Proportions of cells with diploid (G0 or G1 phase), tetraploid (G2 or M phase) or intermediate-state (S phase) DNA contents were compared to those of vehicle-treated cells. Cell cycle phases were used to designate the position of cell cycle arrest. Percentage changes of diploid, S, tetraploid populations, compared to those of vehicle-treated cells, were listed in parentheses.
ND: Not Determined

### REFERENCES

All references cited herein and throughout the specification are herein incorporated by reference in their entirety.
1. Linden, J. Molecular approach to adenosine receptors: receptor-mediated mechanisms of tissue protection. Annu Rev Pharmacol Toxicol, 41: 775-787, 2001.
2. Gordon, J. L. Extracellular ATP: effects, sources and fate. Biochem J, 233: 309-319, 1986.
3. Ralevic, V., Milner, P., Kirkpatrick, K. A., and Burnstock, G. Flow-induced release of adenosine 5′-triphosphate from endothelial cells of the rat mesenteric arterial bed. Experientia, 48: 31-34, 1992.
4. Grierson, J. P. and Meldolesi, J. Shear stress-induced [Ca2+]i transients and oscillations in mouse fibroblasts are mediated by endogenously released ATP. J Biol Chem, 270: 4451-4456, 1995.
5. Ferguson, D. R., Kennedy, I., and Burton, T. J. ATP is released from rabbit urinary bladder epithelial cells by hydrostatic pressure changes—a possible sensory mechanism? J Physiol, 505 (Pt 2): 503-511, 1997.
6. Vizi, E., Huszar, E., Csoma, Z., Boszormenyi-Nagy, G., Barat, E., Horvath, I., Herjavecz, I., and Kollai, M. Plasma adenosine concentration increases during exercise: a possible contributing factor in exercise-induced bronchoconstriction in asthma. J Allergy Clin Immunol, 109: 446-448, 2002.
7. Decking, U. K., Schlieper, G., Kroll, K., and Schrader, J. Hypoxia-induced inhibition of adenosine kinase potentiates cardiac adenosine release. Circ Res, 81: 154-164, 1997.
8. Olah, M. E. and Stiles, G. L. Adenosine receptor subtypes: characterization and therapeutic regulation. Annu Rev Pharmacol Toxicol, 35: 581-606, 1995.
9. Klinger, M., Freissmuth, M., and Nanoff, C. Adenosine receptors: G protein-mediated signalling and the role of accessory proteins. Cell Signal, 14: 99-108, 2002.
10. Tey, H. B., Tan, C. H., and Khoo, H. E. Modulation of DNA synthesis via adenosine receptors in human epidermoid carcinoma (A431) cells. Biofactors, 4:161-165, 1994.
11. Tey, H. B., Khoo, H. E., and Tan, C. H. Adenosine modulates cell growth in human epidermoid carcinoma (A431) cells. Biochem Biophys Res Commun, 187: 1486-1492, 1992.
12. Colquhoun, A. and Newshohne, E. A. Inhibition of human tumour cell proliferation by analogues of adenosine. Cell Biochem Funct, 15: 135-139, 1997.
13. Yao, Y., Sei, Y., Abbracchio, M. P., Jiang, J. L., Kim, Y. C., and Jacobson, K. A. Adenosine A3 receptor agonists protect HL-60 and U-937 cells from apoptosis induced by A3 antagonists. Biochem Biophys Res Commun, 232: 317-322, 1997.
14. Kohno, Y., Sei, Y., Koshiba, M., Kim, H. O., and Jacobson, K. A. Induction of apoptosis n HL-60 human promyelocytic leukemia cells by adenosine A(3) receptor agonists. Biochem Biophys Res Commun, 219: 904-910, 1996.
15. Fishman, P., Bar-Yehuda, S., and Vagman, L. Adenosine and other low molecular weight factors released by muscle cells inhibit tumor cell growth. Cancer Res, 58: 3181-3187, 1998.
16. Bast, R. e. a. Section 31. In: Cancer Medicine, 5 edition, Vol. 5th ed.: BC Decker Inc, 2000.
17. Park, W. C. and Jordan, V. C. Selective estrogen receptor modulators (SERMS) and their roles in breast cancer prevention. Trends Mol Med, 8: 82-88, 2002.
18. Group, E. B. C. T. C. Tamoxifen for early breast cancer: an overview of the randomised trials. Lancet, 351: 1451-1467, 1998.
19. Couse, J. F. and Korach, K. S. Estrogen receptor null mice: what have we learned and where will they lead us? Endocr Rev, 20: 358-417, 1999.
20. Hall, J. M., Couse, J. F., and Korach, K. S. The multifaceted mechanisms of estradiol and estrogen receptor signaling. J Biol Chem, 276: 36869-36872, 2001.
21. Altucci, L., Addeo, R., Cicatiello, L., Dauvois, S., Parker, M. G., Truss, M., Beato, M., Sica, V., Bresciani, F., and Weisz, A. 17beta-Estradiol induces cyclin D1 gene transcription, p36D1-p34cdk4 complex activation and p105Rb phosphorylation during mitogenic stimulation of G(1)-arrested human breast cancer cells. Oncogene, 12: 2315-2324, 1996.
22. Watts, C. K., Sweeney, K. J., Warlters, A., Musgrove, E. A., and Sutherland, R. L. Antiestrogen regulation of cell cycle progression and cyclin D1 gene expression in MCF-7 human breast cancer cells. Breast Cancer Res Treat, 31: 95-105, 1994.
23. Dubik, D., Dembinski, T. C., and Shiu, R. P. Stimulation of c-myc oncogene expression associated with estrogen-induced proliferation of human breast cancer cells. Cancer Res, 47: 6517-6521, 1987.
24. Dubik, D. and Shiu, R. P. Mechanism of estrogen activation of c-myc oncogene expression. Oncogene, 7: 1587-1594, 1992.
25. Foster, J. S and Wimalasena, J. Estrogen regulates activity of cyclin-dependent kinases and retinoblastoma protein phosphorylation in breast cancer cells. Mol Endocrinol, 10: 488-498, 1996.
26. Cariou, S., Donovan, J. C., Flanagan, W. M., Milic, A., Bhattacharya, N., and Slingerland, J. M. Down-regulation of p21WAF1/CIP1 or p27Kip1 abrogates antiestrogen-mediated cell cycle arrest in human breast cancer cells. Proc Natl Acad Sci USA, 97: 9042-9046, 2000.
27. Foster, J. S., Henley, D. C., Bukovsky, A., Seth, P., and Wimalasena, J. Multifaceted regulation of cell cycle progression by estrogen: regulation of Cdk inhibitors and Cdc25A independent of cyclin D1-Cdk4 function. Mol Cell Biol, 21: 794-810, 2001.
28. Podhajcer, O. L., Resnicoff, M., Bover, L., Medrano, E. E., Slavutsky, I., Larripa, I., and Mordoh, J. Effect of estradiol and tamoxifen on the anchorage-independent growth of the subpopulations derived from MCF-7 breast carcinoma cells: cytogenetic analysis of the stem cell subpopulation. Exp Cell Res, 179: 58-64, 1988.
29. Horwitz, K. B., Koseki, Y., and McGuire, W. L. Estrogen control of progesterone receptor in human breast cancer: role of estradiol and antiestrogen. Endocrinology, 103: 1742-1751, 1978.
30. Manni, A., Wright, C., and Buck, H. Growth factor involvement in the multihormonal regulation of MCF-7 breast cancer cell growth in soft agar. Breast Cancer Res Treat, 20: 43-52, 1991.
31. Fishman, P., Bar-Yehuda, S., Ohana, G., Pathak, S., Wasserman, L., Barer, F., and Multani, A. S. Adenosine acts as an inhibitor of lymphoma cell growth: a major role for the A3 adenosine receptor. Eur J Cancer, 36: 1452-1458, 2000.
32. Fishman, P., Bar-Yehuda, S., Madi, L., and Cohn, I. A3 adenosine receptor as a target for cancer therapy. Anticancer Drugs, 13: 437-443, 2002.
33. Brambilla, R., Cattabeni, F., Ceruti, S., Barbieri, D., Franceschi, C., Kim, Y. C., Jacobson, K. A., Klotz, K. N., Lohse, M. J., and Abbracchio, M. P. Activation of the A3 adenosine receptor affects cell cycle progression and cell growth. Naunyn Schmiedebergs Arch Pharmacol, 361: 225-234, 2000.
34. Fredholm, B. B., AP, I. J., Jacobson, K. A., Klotz, K. N., and Linden, J. International Union of Pharmacology. XXV. Nomenclature and classification of adenosine receptors. Pharmacol Rev, 53: 527-552, 2001.
35. Kim, S. G., Ravi, G., Hoffmann, C., Jung, Y. J., Kim, M., Chen, A., and Jacobson, K. A. p53-Independent induction of Fas and apoptosis in leukemic cells by an adenosine derivative, C1-IB-MECA. Biochem Pharmacol, 63: 871-880, 2002.
36. Schrier, S. M., van Tilburg, E. W., van der Meulen, H., Ijzerman, A. P., Mulder, G. J., and Nagelkerke, J. F. Extracellular adenosine-induced apoptosis in mouse neuroblastoma cells: studies on involvement of adenosine receptors and adenosine uptake. Biochem Pharmacol, 61: 417-425, 2001.
37. Barbieri, D., Abbracchio, M. P., Salvioli, S., Monti, D., Cossarizza, A., Ceruti, S., Brambilla, R., Cattabeni, F., Jacobson, K. A., and Franceschi, C. Apoptosis by 2-chloro-2′-deoxy-adenosine and 2-chloro-adenosine in human peripheral blood mononuclear cells. Neurochem Int, 32: 493-504, 1998.
38. Gao, Z., Li, B. S., Day, Y. J., and Linden, J. A3 adenosine receptor activation triggers phosphorylation of protein kinase B and protects rat basophilic leukemia 2H3 mast cells from apoptosis. Mol Pharmacol, 59: 76-82, 2001.
39. Neuman, E., Ladha, M. H., Lin, N., Upton, T. M., Miller, S. J., DiRenzo, J., Pestell, R. G., Hinds, P. W., Dowdy, S. F., Brown, M., and Ewen, M. E. Cyclin D1 stimulation of estrogen receptor transcriptional activity independent of cdk4. Mol Cell Biol, 17: 5338-5347, 1997.
40. Romieu-Mourez, R., Landesman-Bollag, E., Seldin, D. C., Traish, A. M., Mercurio, F., and Sonenshein, G. E. Roles of IKK kinases and protein kinase CK2 in activation of nuclear factor-kappaB in breast cancer. Cancer Res, 61: 3810-3818, 2001.
41. Cataldo, L. M., Zhang, Y., Lu, J., and Ravid, K. Rat NAP1: cDNA cloning and upregulation by Mp1 ligand. Gene, 226: 355-364, 1999.
42. Sun, S. and Ravid, K. Role of a serine/threonine kinase, Mst1, in megakaryocyte differentiation. J Cell Biochem, 76: 44-60, 1999.
43. Wang, Z., Zhang, Y., Lu, J., Sun, S., and Ravid, K. Mp1 ligand enhances the transcription of the cyclin D3 gene: a potential role for Sp1 transcription factor. Blood, 93: 4208-4221, 1999.
44. Pianetti, S., Guo, S., Kavanagh, K. T., and Sonenshein, G. E. Green tea polyphenol epigallocatechin-3 gallate inhibits Her-2/neu signaling, proliferation, and transformed phenotype of breast cancer cells. Cancer Res, 62: 652-655, 2002.
45. Robak, T. The role of nucleoside analogues in the treatment of chronic lymphocytic leukemia-lessons learned from prospective randomized trials. Leuk Lymphoma, 43: 537-548, 2002.
46. Williams, W., Craver, R. D., Correa, H., Velez, M., and Gardner, R. V. Use of 2-chlorodeoxyadenosine to treat infantile myofibromatosis. J Pediatr Hematol Oncol, 24: 59-63, 2002.
47. Koc, Y., Urbano, A. G., Sweeney, E. B., and McCaffrey, R. Induction of apoptosis by cordycepin in ADA-inhibited TdT-positive leukemia cells. Leukemia, 10: 1019-1024, 1996.

1. A method for downregulating estrogen receptors in a population of cells expressing an estrogen receptor comprising delivering to said population of cells an effective amount of at least one adenosine analog and a pharmaceutically acceptable carrier to down-regulate estrogen receptor levels.

2. The method of claim 1, wherein the population of cells comprise malignant cells.

3. The method of claim 2, wherein the malignant cells are breast cancer cells.

4. The method of claim 2 or 3, wherein the population of cells are estrogen receptor alpha positive.

5. The method of claim 1, wherein the adenosine analog is an adenosine A3 receptor agonist.

6. The method of claim 5, wherein the adenosine analog is N⁶-(3-iodobenzyl)adenosine-5′-N-methyluronamide or a derivative thereof.

7. The method of claim 5, wherein the adenosine analog is 2-chloro-adenosine or a derivative thereof.

8. The method of claim 1, wherein the downregulation of estrogen receptor levels results from decrease in estrogen receptor transcription.

9. The method of claim 1, wherein at least one cell in the population of cells is or has become resistant to (Z)1,2-diphenyl-1-[4-[2-(dimethylamino) ethoxy]phenyl]-1-butene, 4-OH-(Z)1,2-diphenyl-1-[4-[2-(dimethylamino) ethoxy]phenyl]-1-butene, raloxifene, or N-(n-butyl)-1-[3,17β-dihydroxyestra-1,3,5(10)-trien-7α-yl]N— methylundecanamide or a derivative thereof.

10. The method of claim 1, wherein the at least one adenosine analog and a pharmaceutically acceptable carrier to decrease estrogen receptor levels are delivered before, after or simultaneously with (Z) 1,2-diphenyl-1-[4-[2-(dimethylamino) ethoxy]phenyl]-1-butene, 4-OH-(Z) 1,2-diphenyl-1-[4-[2-(dimethylamino) ethoxy]phenyl]-1-butene, raloxifene, or N-(n-butyl)-1′-[3,17β-dihydroxyestra-1,3,5(10)-trien-7α-yl]N— methylundecanamide or other estrogen receptor regulating pharmaceutical, or a combination thereof.

11. The method of claim 1, wherein at least one cell in the population of cells is growing via anchorage-independent manner.

12. The method of claim 1, wherein at least one cell in the population of cells is growing via anchorage-dependent manner.

13. The method of claim 4, wherein the population of cells comprise at least one cell which is estrogen receptor alpha positive.

14. A method of suppressing cell cycle and/or cellular growth in a population of cells comprising delivering to the cell population an effective amount to downregulate estrogen receptor levels, at least one adenosine analog and a pharmaceutically acceptable carrier.

15. The method of claim 14, wherein the population of cells comprises malignant cells.

16. The method of claim 15, wherein the malignant cells are breast cancer cells.

17. The method of claim 15, wherein the malignant cells are ovarian cancer cells.

18. A method of treating an individual affected with malignant cell growth in a tissue or plurality of tissues expressing estrogen receptors, the method comprising administering to the individual a sufficient amount of an adenosine agonist to downregulate estrogen receptors in a cell population in the tissue or plurality of tissues and a pharmaceutically acceptable carrier.

19. The method of claim 18, wherein the malignant cell growth is breast cancer.

20. The method of claim 18, wherein the malignant cell growth is ovarian cancer.

21. The method of claim 18, wherein the malignant cell growth is anchorage-independent.

22. The method of claim 18, wherein at least one of the estrogen receptors expressed by the cell population is mutated or truncated.

23. The method of claim 18, wherein the estrogen receptor is estrogen receptor alpha.

24. The method of claim 23, wherein the malignant cell growth is breast cancer or ovarian cancer.

25. A method of identifying a compound suitable for treating malignant cell growth in a tissue which expresses estrogen receptors, the method comprising measuring the amount of estrogen receptor expression in a cell, administering an adenosine analog to the cell, and measuring the expression of estrogen receptor after administration of the adenosine analogue, wherein reduction in the amount of estrogen receptor in the cell after administration of the adenosine analogue indicates identification of a compound suitable for treating malignant cell growth.

26. The method of claim 25, wherein the malignant cell growth is beast cancer or ovarian cancer.

27-32. (canceled)

33. A kit for downregulating estrogen receptors in a population of breast and/or ovarian cancer cells comprising in a container at least one adenosine analog capable of downregulating estrogen receptors, wherein the adenosine analog is an adenosine A3 receptor agonist selected from the group consisting of N⁶-(3-iodobenzyl)adenosine-5′-N-methyluronamide, 2-chloro-adenosine and a derivative of N⁶-(3-iodobenzyl)adenosine-5′-N-methyluronamide, 2-chloro-adenosine, in the population of cells in a pharmaceutically acceptable carrier in a vial or tube, a means for detecting downregulation of estrogen receptors in the population of cells, and an instruction manual exemplifying how to measure estrogen receptor downregulation using the means provided in the kit.

34-36. (canceled)

37. A kit for detecting compounds capable of downregulating estrogen receptors in a population of cells comprising:
a population of test cells expressing estrogen receptors in a suitable cell growth medium or freezing medium or storage medium;
a standard adenosine analog capable of downregulating estrogen receptors in the population of test cells as powder or in a suitable buffer with known concentration;
a means for detecting estrogen downregulation in the test cell population, wherein the test cell population comprises malignant cells, wherein the malignant cells are breast and/or ovarian cancer cells and wherein the malignant cells are resistant to a compound selected from the group consisting of (Z) 1,2-diphenyl-1-[4-[2-(dimethylamino) ethoxy]phenyl]-1-butene, 4-OH-(Z) 1,2-diphenyl-1-[4-[2-(dimethylamino) ethoxy]phenyl]-1-butene, raloxifene, or N-(n-butyl)-11-[3,17β-dihydroxyestra-1,3,5(10)-trien-7α-yl]N-methylundecanamide; and
an instruction manual outlining exemplary cell growth conditions to detect downregulation of estrogen receptors in the test cell population using the standard adenosine analog, wherein the standard adenosine analog is selected from the group consisting of adenosine A3 receptor agonist, N⁶-(3-iodobenzyl)adenosine-5′-N-methyluronamide and 2-chloro-adenosine.

38-47. (canceled)
